(19) United States
(12) Patent Application Publication
(10) Pub. No.: US 2003/0148274 A9
(43) Pub. Date: Aug. 7, 2003

(54) Identification of neural defects associated with the nucleosomal assembly protein 112 gene

(76) Inventors: Ute Rogner, Paris (FR); Demetri Spyropoulos, James Island, SC (US); Claire Rougeulle, Paris (FR); Philip R. Avner, Paris (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 09/847,665

(22) Filed: May 3, 2001

Related U.S. Application Data
(60) Provisional application No. 60/202,111, filed on May 5, 2000.

(51) Int. Cl. C12Q 1/68; C07H 21/04

(52) U.S. Cl. 435/6; 536/24.3

(57) ABSTRACT

A method for screening neural system defects in a human comprises: (A) providing chromosomal material from the human; (B) detecting a modification of the NAP1L2 gene in the chromosomal material, wherein the modification is selected from a) substitution, b) deletion, c) frame-shift, or d) insertion that causes a loss of biological function in the NAP1L2 gene; and (C) correlating the modification of the gene with a potential for a neural system defect. The method can also be practiced with the mouse Nap1l2 gene.

### CROSS-REFERENCE TO RELATED APPLICATIONS

This application is based on and claims the benefit of U.S. Provisional Application Ser. No. 60/202,111, filed May 5, 2000 (attorney docket no. 03495.6048) The entire disclosure of this application is relied upon and incorporated by reference herein.

### BACKGROUND OF THE INVENTION

This invention relates to the discovery of a link between defects in development of the central nervous system of a mammal and mutations in a gene, which result in a loss of biological function of protein encoded by the gene. Mutated forms of the gene, the RNA, and the protein it encodes are useful in diagnosis of predisposition to genetic defects.

Neurulation is a complex process of histogenesis involving the precise temporal and spatial organization of gene expression. Amongst the molecular components necessary for neurulation are proneural genes determining primary cell fate, neurogenic genes involved in the lateral inhibition pathway, and genes controlling the frequency of mitotic events^{1,2}. This underlying complexity is reflected in the aetiology and genetics of human neural tube defects (NTDs), which are of both multifactorial and multigenic origin³. Similar complexity is observed in mouse models of NTDs where genes affecting cell fate such as Sonic hedgehog⁴ and the Pax genes⁵, genes encoding cell adhesion molecules like cadherins⁶, and genes affecting neural cell division, such as p53 and Nfl⁷, have all been implicated in the process of neurulation.

One family of genes, which has been implicated in the control of mitotic events^{8,9}, is the NAP-1 family. The NAP-1 protein was first identified in Xenopus laevis¹⁰ and homologous proteins subsequently isolated from drosophila¹¹, yeast¹² and man⁹. NAP-1 and NAP-1 like proteins have been shown to transfer nucleosome units to naked DNA¹⁰, to stimulate transcription factor binding to nucleosomal DNA¹³, and to act as core histone shuttle implicated in the transport histones from the cytoplasm to the nucleus¹⁴. Control of mitotic events may depend on the role of NAP-1 and NAP-1 like proteins in chromatin assembly and remodeling or more directly through their binding to cyclins, which is mediated by a domain also found in the tumor associated SET proteins¹⁵.

The recently isolated murine X-linked Nap1l2 (Bpx) and its human homologue NAP1L2 (BPX) have a highly restricted pattern of expression, being expressed exclusively in the nervous system¹⁶. In this respect, NAP1L2 and the X-linked brain-specific NAP1L3¹⁷ differ from the ubiquitously expressed NAP1L1 and NAP1L4 genes. The limited expression pattern of these genes suggests a particular and specialized function, possibly through an effect on nucleosome assembly or cell cycle regulation, specific to neural function.

Neural tube defects occur with a frequency of 3.5/1000 births. There is a need in the art for the identification of genes associated with defects in development of the central nervous system. In particular, there is a need for diagnostic tests and biological materials to identify predisposition to developmental defects.

### SUMMARY OF THE INVENTION

This invention aids in fulfilling these needs in the art. More particularly, this invention relates to the discovery of the role of Nap1l2 in mouse development. A targeted deletion of the X-linked Nap1l2 gene in male ES cells, which would be expected to lead to the complete absence of the NAP1L2 protein, was created. In close agreement with the first detectable signs of Nap1l2 expression at day E9.5, the mutation resulted in embryonic lethality from mid-gestation onwards. Surviving embryos derived from ES cell-morula aggregates exhibited neural tube closure defects, associated with a marked overproduction of neuronal tissue.

This invention shows that Nap1l2 plays an essential role in the development of the nervous system and suggests a putative role for it in the control of cell proliferation and differentiation processes. Aberrant cell cycle regulation and differentiation may, therefore, be one of the mechanisms underlying certain neural tube defects (NTDs). This invention also identifies the human NAP1L2 gene as a gene for certain X-linked and spontaneous forms of these disorders.

One embodiment of this invention relates to a method for screening neural system defects in the mammal and especially in the human. The method comprises: (A) providing genomic material from the human; (B) detecting a modification of the NAP1L2 gene in the genomic material, wherein the modification is selected from a) substitution, b) deletion, c) frame-shift, d) insertion aberent or e) altered epigenetic control that causes a loss of biological function in the NAP1L2 gene; and (C) corre-lating the modification of the gene with a potential for a neural system defect. In a preferred embodiment, the modification in the NAP1L2 gene is detected by hybridization with a labeled probe, such as a probe. of SEQ ID NO:3 or a fragment thereof. The modification can be detected, for example, by (A) amplification of the genomic material using PCR; (B) sequencing the material to detect the modification of the nucleotide sequence; and (C) correlating the modification of the gene with a potential for neural system defects. The modification can be detected by quantification of the transcript using PCR or Northern Blot.

In another embodiment, the invention provides a method for screening neural system defects in a human, this method comprises: (A) providing biological material from the human; (B) detecting the absence, inappropriate, or modified expression of NAP1L2 gene product using labeled antibodies to the gene product; and (C) correlating the absence, inappropriate, or modified expression with a potential for neural system defects. The antibodies can be polyclonal or monoclonal.

The neural system defect can result from a failure of, or incomplete, neural tube closure, incomplete neural tube closure resulting in spina bifida, incomplete neural tube closure resulting in anencephaly, neural system defect relating to an inappropriate proliferation of surface ectoderm-derived cells, neural defect resulting in a loss of brain structure, neural system defect resulting from disorganization of brain structures, neural system defect resulting from inappropriate control of nucleosome activity in neurons, neural system defect resulting from inappropriate control of the cell cycle in neurons, inappropriate differentiation of neurons, inappropriate maintenance of neurons, neural system defect resulting from inappropriate control of cyclins and associated proteins, NSD resulting from inappropriate control of kinases, neural system defect characterized by an X-chromosome linked disorder, NSD characterized by methylation deficiencies, neural system defect resulting from inappropriate control of transcription factors binding to and co-activators to DNA, neural system defect resulting from inappropriate control of histones, including their modification e.g., acetylation and phosphorylation, or neural system defect resulting from inappropriate shuttle of histones from the cytoplasm to the nucleus, inappropriate integration of histones into chromatin.

This invention also provides a recombinant chromosome comprising a polynucleotide containing a nucleotide sequence, wherein the sequence includes at least one modification of the NAP1L2/Napsll gene, wherein the modification is selected from a) substitution, b) deletion, c) frame-shift, d) aberrent insertion, e) altered epigenetic control, or f) site-directed mutagenesis that causes a loss of biological function in the NAP1L2 gene.

This invention also provides a neural cell containing the recombinant chromosome. The chromosome can be a neural cell chromosome. The neural cell can be derived from an immortal cell line or a stem cell line or a neural stem cell line (including human), and in one embodiment the chromosome can be a stem cell chromosome. The mouse stem cell can be derived, for example, from stem cell line CK35, stem cell line 5b17, or stem cell line 8b21. A β-gal gene can be substituted for the NAP1L2/Nap1l2 gene, wherein the stem cell also contains genetic material for selection in selection media.

This invention further provides a method of simultaneously monitoring the expression (e.g. detecting and/or quantifying the expression) of the Nap1l2 gene or the NAP1L2 gene. The method involves providing a pool of target nucleic acids comprising mRNA transcripts of one or more of these genes, or nucleic acids derived from the mRNA transcripts, hybridizing the pool of nucleic acids to oligonucleotide probes, wherein the oligonucleotide probes are complementary to the mRNA transcripts or nucleic acids derived from the mRNA transcripts, and quantifying the hybridized nucleic acids. The pool of target nucleic acid can be one on which the concentration of the target nucleic acids (mRNA transcripts) or nucleic acids derived from the mRNA transcripts is proportional to the expression levels of the Nap1l2 or NAP1L2 genes. Microfabricated arrays of large numbers of different oligonucleotide probes (e.g. DNA chips) may effectively be used to detect the presence or absence of the target nucleic acid sequences and to quantify the relative abundance of the target sequences in a complex nucleic acid pool.

Further, this invention provides a chimeric mouse having at least one cell, which is progeny of the stem cells of the invention. The chimeric mouse can be derived from a morula of a CD-1 mouse, such as a morula of a C57BL/6 mouse.

This invention also provides a recombinant neural cell comprising a vector comprising the NAP1L2 gene. The NAP1L2 gene can be under control of a neural-specific promoter. The neural cell can be from a wild-type animal. A NAP1L2 gene can be modified, wherein the modification is selected from a) substi-tution, b) deletion, c) frame-shift, d) insertion, or e) site-directed mutagenesis that causes a loss of biological function in a NAP1L2 gene. The NAP1L2 gene of the native cell can be altered through a naturally occurring mutation.

Another embodiment of this invention involves the discovery of the promoter of the Nap1l2 gene in SEQ ID NO:1, and the discovery of the NAP1L2 gene in SEQ ID NO:4. Further, this invention provides vectors and recombinant cells containing either one or both of these promoters.

Genomic Structure of the NAP1L2 Gene

NAP1L2 is an intronless gene with an open reading frame of 1383 bp, followed by a 796 bp lor non-translated 3′-region (Rougeulle and Avner, 1996). (cf Ref. 16). The untranslated 5′ region of NAP1L2 extends over the first 312 bp of the cDNA sequence. In this analysis, we characterized an additional 1208 bp of repeat-free genomic sequence lying upstream of the putative transcription start site, which likely contains the promoter region of NAP1L2. See FIG. 12.

Further, this invention provides a method of making a recombinant neural cell comprising: (A) providing a neural cell; (B) modifying a NAP1L2 gene or the promoter of the NAP1L2 gene in the neural cell, wherein the modification is selected from a) substitution, b) deletion, c) frame-shift, and d) insertion that causes a loss of biological function in the gene; and (C) selecting modified cells.

In addition, this invention provides a method of screening for therapeutic compounds comprising: (A) providing a cell of the invention; (B) introducing to the cell a compound to be screened; and (C) correlating change in the proliferation or differentiation of cells with the activity of the compound. The change in proliferating cells can be a control of cancer. For example, the cancer can be in neuronal tissue or stem cells capable of giving rise to neuronal tissue and epithelium.

The method of screening for therapeutic compounds is also provided. The method comprises: (A) providing an animal or animal embryo of the invention; (B) introducing to the animal or animal embryo a compound to be screened; and (C) correlating a change in the development and maturation of the nervous system with the activity of the compound. The change in the nervous system can be alteration or prevention of spina bifida, for example, or an alteration or prevention of anencephaly.

Besides diagnosis, this invention is also directed to:

the use of the promoter sequence for expression or the regulation of the expressions of endogenous or exogenous genes in neural cells, preferably post-mitotic neural cells, especially for therapeutic purposes;

a method for obtaining such regulation is described in the European Patent EP 419621 (Institut Pasteur).

the use of the gene to predict tumor of neural origin and to prevent tumor development due to overproduction of neural cells caused by the non expression of NAP1L2 gene; and

the use of this gene in genetic therapy of neurodegenerative diseases, such as Parkinsons or Alzheimers diseases, or accidents, especially by development of new neural networks.

The invention provides these same embodiments relating to the mouse Nap1l2 gene instead of the human NAP1L2 gene.

### BRIEF DESCRIPTION OF THE DRAWINGS

This invention will be described in detail with reference to the drawings in which:

FIG. 1 shows localization of Nap1l2 mRNAs in the mouse nervous system by in situ hybridization with oligonucleotide probes.

a, Bright-field photographs of film autoradiograms showing Nap1l2 expression at different developmental stages. The expression domain covers all structures of the nervous system, the neural tube, and the peripheral ganglia.

b, Dark-field microphotographs of emulsion autoradiograms showing details of Nap1l2 expression in the nervous system. Left panel, Expression at the lumbar level in the E11.5 spinal cord and peripheral ganglia. Note the more extensive labeling in the ventral part, due to the ventro-dorsal differentiation gradient, also visible at E15.5 on the film autoradiogram. Right panel, Expression in the E11.5 superior colliculus. Here again the ventricular zone is devoid of labeling.

c, Bright-field photographs of film autoradiograms showing Nap1l2 expression in the adult mouse brain. Expression is widespread throughout the brain. However, the intensity of staining is not correlated with cell density, suggesting variable expression. Note the strong labeling of the mammillary bodies. Abbrv: V, Trigeminal ganglion; IX/X, ganglionic complex of the IX and X nerves; Cer, Cerebellum; Cx, Cortex; drg dorsal root ganglion; Hip, Hippocampus; Mb, Mammillary bodies; Me5, Mesencephalon; ne neuroepithelium; Pr, Prosencephalon; Rh, Rhombencephalon; SC, Spinal Cord; St, Striatum; Th, Thalamus.

FIG. 2 shows construction of a Nap1l2 knockout.

a, Restriction pattern of genomic DNA.

b, Deletion of Nap1l2 and insertion of the lacZneo and HSV-tk cassettes in the plasmid construction.

c, Restriction pattern of the ES cell lines 5b17 and 8b21 after homologous recombination.

FIG. 3 shows that ES cells from a mutant ES cell line 5617 differentiate in vitro into neurons. Photos of immunofluorescence using anti-nestin antibodies are shown in panels a, b, c. Comparison of a, the usual cell line Ck 35 dob, the mutant cell line 5B17 on day nine of in vitro differentiation e, lac 2 positive cells (see arrows) are found within the nestin position cell population.

FIG. 4 shows average numbers of chimeric, retarded, and resorbed embryos at different time points during mouse development obtained in morula aggregation experiments. Numbers are given as percentages of total embryos recovered. Embryos obtained from the CD-1 and C57BL/6 experiments are listed separately. Each foster mother was implanted with 8-12 morulas.

FIG. 5 depicts mutant chimeric embryos obtained from morula aggregation experiments. The day E17.5 embryo (CD-1 morula aggregate) is one of two embryos found in the same experiment that exhibited anencephaly. The day E14.5 embryo is a high percentage chimeric CD-1 aggregate with dark eye pigmentation that shows detached surface ectoderm. Day E12.5: In the experiments using CD-1 morulas, 14 non-resorbed chimeric embryos were found, including six that displayed lacZ staining predominantly along the dorsal midline. The day E12.5 embryos shown here are representative of the ectoderm defects found (arrows):

a, b, and c, open neural tube, d, hindbrain ablation, e, exposed telencephalon, a control embryo is show on the right site of photo d. The apparently normal E10.5 chimera has lacZ staining along its entire dorsal length. The two apparently normal E9.5 chimeras show lacZ staining restricted to the very caudal tip of the tail.

FIG. 6 shows sections of lacZ stained embryos with specific phenotypes.

a, Section of a day E12.5 embryo with an open neural tube defect (arrow) in the upper thoracic region.

b, Strong rearrangements of the brain of a day E12.5 embryo. The position of the rhombencephalon is indicated by rh.

c, Exposed neural tube (E12.5, compare to A).

d, Exposed neural tissue (arrow), of the brain (E12.5, compare to B).

e, Detached surface ectoderm (se) and spinal cord (sc) at day E13.5.

f, Overproduction of surface ectoderm (arrow) at day E13.5. Note also the lacZ staining in the virbrissa.

g, Possible necrosis (arrow) in the brain of a chimeric day E14.5 embryo.

h, Overproduction of surface ectoderm and lacZ staining in the underlying mesenchyme (arrow) at day E14.5.

FIG. 7 depicts the sequence of clone Bpx promoter murine SpeI-SalI fragment (SEQ ID NO:1) containing the Nap1l2 promoter.

FIG. 8 depicts the sequence of Bpx murine cDNA identical to genomic DNA (SEQ ID NO:2).

FIG. 9 depicts the sequence of human cDNA identical to genomic DNA (SEQ ID NO:3).

FIG. 10 depicts the human BPX 5′ region containing the NAP1L2 gene promoter (SEQ ID NO:4).

FIG. 11 shows the results of the study of localization of the NAP1L2 gene product in embryonal carcinoma cells. P19 cells transfected with the expression vector pEGFP express GFP-NAP1L2 fusion proteins under the control of the CMV promoter (a to c). The cells are counterstained with DAPI. a, NAP1L2 localises to the cytoplasm of cells arrested in G1 phase, (growth in medium with 0.5% FCS for 72 hours); and b, to the nucleus of cells arrested in S-phase (10⁻⁷ M methotrexate for 24 h); c, NAP1L2 localises to the condensed chromatin in cells undergoing nuclear fragmentation, the lower panel shows the DAPI counterstaining of c. Expression of the GFP fusion protein under the control of the endogenous Nap1l2 promoter (d to g), d, Cell expressing the NAP1L2 fusion protein during or after S-phase show chromatin condensation; e, BrdU staining of d; f, DAPI staining of d; g merge of d, e and f.

FIG. 12 is a diagram of the genomic structure of the NAP1L2 gene.

### DETAILED DESCRIPTION OF THE INVENTION

It has been discovered that Nap1l2, a NAP-1 like gene, is involved in the development of the neural tube, and that deletion of Nap1l2 results in embryonic lethality from mid-gestation of embryogenesis onwards. Surviving embryos show severe neural tube closure defects, which closely resemble spina bifida and/or anencephaly in humans.

In situ hybridization studies have indicated that Nap1l2 is almost exclusively expressed in post-mitotic neurons of both the central and peripheral nervous system. Expression of Nap1l2 is initially detected at day E9.5 of mouse development (FIG. 5) compatible with the first signs of altered embryogenesis in chimeric embryos deleted for the Nap1l2 gene. The effects of deletion of the Nap1l2 gene becomes progressively more severe as embryogenesis proceeds, culminating in extensive embryo resorption between days E12.5 and E14.5 and an absence of high level chimerism in recovered newborns. Examination of the exceptional viable chimeras recovered at various embryonic stages, post day E9.5, suggests that this lethality is due to a defect in the proliferation of ectodermally derived cells and associated abnormal histogenesis, primarily affecting the spinal cord and the brain. The severity of spinal cord defects appears to correlate well with the extent of chimerism as demonstrated by lacZ expression.

Since an increase in the numbers of certain lacZ negative cells in the chimeric embryos (FIG. 6h) was observed, it is likely that the Nap1l2 mutation is not fully cell-autonomous. The absence of Nap1l2 in one cell could, therefore, interfere with the proliferation of neighboring cells by a cell-cell interactive process. Extensive cell-cell interaction, involving neurotrophic factors, is, for example, known to be involved in neurogenesis and the survival of neurons clearly depends not only on their own cell cycle, but also on the interaction with other neurons¹⁹. The Otx2 gene has, for example, recently been shown to act both cell autonomously and non-cell autonomously to regulate gene expression in neuroectoderm cells²⁰.

Since lacZ staining was also found in ganglia of E12.5 embryos and the intestinal submucosal plexuses of E12.5 and E17.5 chimeric embryos without there being any obvious consequence for the development of these tissues, the conclusion is that an absence of Nap1l2 function does not obligatorily lead to observable changes in all tissues in which it is normally expressed. The pattern of lacZ expression observed in the chimeric embryos was similarly suggestive of potential Nap1l2 expression in vibrissae of E12.5 and E13.5 embryos (FIG. 6f) and some muscles of E12.5 embryos (FIGS. 5, a to c), again without detectable alteration of these tissues. In these cases, however, Nap1l2 expression could not be confirmed by in situ hybridization studies on normal embryos.

Transcription of Nap1l2 was clearly observed in in vitro differentiated ES cells as established by RT-PCR analysis of wild type ES cells, and by lacZ staining of the Nap1l2 mutant ES cell lines. The observations made in vitro of the Nap1l2 deficient ES cells, which suggest that the Nap1l2 gene is not necessary for the formation and differentiation of post-mitotic neurons, are compatible with the results obtained in vivo in so far that they suggest that Nap1l2 influences and controls the extent of formation and/or proliferation of such neuronal cells rather than the presence of neuronal cells per se (FIG. 6b).

This observation suggests that the Nap1l2 gene is either playing a role in the cell cycle regulation of developing neurons or in the maintenance of their quiescent post-mitotic state. The absence in vivo of the Nap1l2 gene would lead to altered rates of cell division and in the numbers of neural cells formed, which would be disruptive of the normal developmental process implicated in brain development and neural tube closure.

Kerszberg and Changeux have recently formalized such observations and pointed out that an excessive mitotic rate in neural tissues would, by altering the normal matching of rates of division of neural and non-neural tissues, lead to an extension of the neural plate and associated problems in bending the larger than normal plate sufficiently to achieve complete closure¹.

Other examples suggestive of the importance of control of cell proliferation in neural tube closure include the HES-1 and Pax-3 genes. Null mutations of the helix-loop-helix factor HES-1 gene, a homologue of E(spl) and hairy, have been shown to be associated with both an excessive development of neural tissue and associated abnormalities in neural tube closure²¹. Likewise, mutations in the Pax-3 gene responsible for the splotch phenotype in the mouse have been associated with both disorganization of the neuroepithelium and differences in the rate of cell division²². In both the splotch mouse, a neural tube defect model for the Wardenburg syndrome in humans, and the curly tail mouse, the expression level of a number of genes that govern the cell cycle has been found to be altered^{23, 24}.

A putative role for Nap1l2 in neural cell fate correlates well with the high degree of similarity that Nap1l2 shows with other NAP paralogues. Conservation is particularly strong in the two known functional domains both of which contain nuclear localization motifs, suggesting that NAP1L2 might well be able to interfere with nucleosome structure or to bind to cyclins, and through such a mechanism, intervene in cell cycle regulation. Interestingly, preliminary data suggests that overexpression of NAPIL2 in either P19 embryocarcinoma or NIE115 neuroblastoma cells can indeed prevent cell growth and lead to apoptosis (data not shown).

Neurulation requires a precise temporal and spatial organization of gene expression, and Nap1l2 may well represent yet one more gene that has to be taken account of in studies of this process^{1, 2}. Such underlying complexity agrees well with the aetiology of human neural tube defects (NTDs), which are considered to be of multifactorial origin involving the combined action of multiple genes and genetic-environmental interactions. Mutations in several different genes, including PAX-1²⁵, MTHFD²⁶, and SHH²⁷, have already been implicated in human NTDs.

Although several X-linked cases of spina bifida with anencephaly^{28, 29} have been reported, in no case has the defect been characterized. Whilst a complete absence of NAP1L2 might be expected to be lethal in humans, one cannot exclude that certain types of mutations might lead to less severe disorders nor predict to what extent the human and mouse syndromes may differ in severity.

In humans, heritability for anencephaly and spina bifida has been estimated to be around 60%³. Extensive variability in penetrance depending on the genetic background also clearly characterizes many of the known mouse NTD mutations, including both curly tail³⁰ and loop-tail³¹. The differences observed in phenotypic penetrance of the Nap1l2 mutation seen on the CD-1 and C57BL/6 backgrounds, may well reflect this complexity. It is interesting in this context that penetrance of the curly tail mutation is particularly high on the C57BL/6 background³⁰.

This invention, which resulted from these experiments and observations, thus involves mutated forms of Nap1l2 and NAP1L2 genes and the resulting mutated polypeptides they encode. The mutated polypeptides are characterized by a loss of biological function, namely, a loss of normal control of neuronal cell proliferation. This loss of biological function can be associated with aberrant nucleosome assembly, cell cycle regulation, or both, by the mutated Nap1l2 or NAP1L2 genes. The mutated polynucleotide can even lead to embryonic lethality from mid-gestation onwards. If mutant embryos survive, they may show extensive surface ectoderm defects, the presence of open neural tubes, and exposed brains. These mutated polypeptides and fragments thereof, as well as the polynucleotides that encode them and the corresponding RNAs, are useful in diagnostic applications.

As used herein, the expression “a loss of biological function” refers to a mutation in a Nap1l2 gene, or fragment thereof or a Nap1l2 gene a fragment thereof, such that the mutant protein or polypeptide encoded by the mutated gene or polynucleotide fragment is characterized by a loss of biological activity as evidenced by, for example, at least one of the following neural system defects in an animal or embryo containing the mutated gene or polynucleotide:

a failure of or incomplete neural tube closure;

spina bifida;

anencephaly

inappropriate proliferation of surface ectoderm-derived cells;

a loss of brain structures;

disorganization of brain structures;

inappropriate control of nucleosome activity in neurons;

inappropriate control of the cell cycle in neurons and neuronal cell precursors;

inappropriate control of cyclins and their interacting partners;

an X-chromosome linked disorder;

inappropriate control of transcription factors binding to DNA;

inappropriate control of histones; and

inappropriate shuttle of histones from the cytoplasm to the nucleus.

The ability of the mutated genes and polynucleotides and the proteins and polypeptides they encode to produce these neural system defects can be determined using the techniques described in the Examples and the Methods set forth hereinafter.

The implications for this invention are widespread. A cDNA encoding mutated Nap1l2 protein has been isolated. This discovery of the cDNA encoding mutated Nap1l2 protein enables construction of expression vectors comprising nucleic acid sequences encoding mutated Nap1l2 and polypeptides, and the corresponding RNAs; host cells transfected or transformed with the expression vectors; biologically inactive Nap1l2 polypeptides and mutated Nap1l2 polypeptides as isolated or purified proteins; and antibodies immunoreactive with mutated Nap1l2 polypeptides. In addition, understanding of the mechanism by which mutated Nap1l2 polypeptides function enables the design of assays to detect substances that affect Nap1l2 protein activity. It will of course be understood that while the various embodiments of this invention may be described with reference to mouse Nap1l2, the invention applies to the same extent to human NAP1L2.

Thus, as used herein, the term “mutated Nap1l2 and NAP1L2 polypeptides” refers to a genus of mutated polypeptides that exhibit a loss of biological function, and that further encompasses mutated proteins and polypeptides described herein, as well as those mutated proteins and polypeptides having a high degree of similarity (at least 90% homology) with such amino acid sequences, and which proteins and polypeptides lack at least one of the biological activities of naturally occurring Nap1l2 and NAP1L2 proteins.

The term “mutated Nap1l2 and NAP1L2 polynucleotides” refers to the polynucleotides that encode the respective mutated Nap1l2 and NAP1l2 polypeptides of the invention, and the RNAs corresponding to these polynucleotides. The mutated polynucleo-tides of the invention contain mutations in the 5′ region of the Nap1l2 and NAP1L2 genes leading to complete absence of the gene product.

The term “purified” as used herein, means that the mutated Nap1l2 and NAP1L2 polypeptides and polynucleotides are essentially free of association with other proteins or polypeptides and polynucleotides, for example, as a purification product of recombinant host cell culture or as a purified product from a non-recombinant source. The term “substantially purified” as used herein, refers to a mixture that contains mutated Nap1l2 and NAP1L2 polypeptides and polynucleotides and is essentially free of association with other proteins or polypeptides and polynucleotides, but for the presence of known proteins that can be removed using a specific antibody, and which substantially purified mutated Nap1l2 and NAP1L2 polypeptides can be used as antigens.

Variants can comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring mutated Nap1l2 and NAP1L2 polypeptide variants are also encompassed by the invention. Examples of such variants are proteins that result from proteolytic cleavage of the mutated Nap1l2 and NAP1L2 polypeptides. Variations attributable to proteolysis include, for example, differences in the termini upon expression in different types of host cells, due to proteolytic removal of one or more terminal amino acids from the mutated Nap1l2 and NAP1L2 polypeptides. Variations attributable to frame shifting include, for example, differences in the termini upon expression in different types of host cells due to different amino acids. Variation can also result from a chemical modification by phosphorylation.

As stated above, the invention provides isolated and purified, or homogeneous, mutated Nap1l2 and NAP1L2 polypeptides, both recombinant and non-recombinant. Variants and derivatives of native mutated Nap1l2 and NAP1L2 polypeptides that can be used as antigens can be obtained by mutations of nucleotide sequences coding for native mutated Nap1l2 and NAP1L2 polypeptides.

Alterations of the native amino acid sequence can be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene, wherein predetermined codons can be altered by substitution, deletion, or insertion. Exemplary methods of making the alterations set forth above are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462, all of which are incorporated by reference.

Nucleic acid sequences or polynucleotides within the scope of the invention include isolated DNA and RNA sequences that hybridize to the native mutated Nap1l2 and NAP1L2 nucleic acids disclosed herein under conditions of moderate or severe stringency, and which encode mutated Nap1l2 and NAP1L2 polypeptides. As used herein, conditions of moderate stringency, as known to those having ordinary skill in the art, and as defined by Sambrook et al. Molecular Cloning: A Laboratory Manual, 2 ed. Vol. 1, pp. 1.101-104, Cold Spring Harbor Laboratory Press, (1989), include use of a prewashing solution for the nitrocellulose filters 5×SSC, 0.5% SDS, 1.0 mM EDTA (pH 8.0), hybridization conditions of 50% formamide, 6×SSC at 42° C. (or other similar hybridization solution, such as Stark's solution, in 50% formamide at 42° C.), and washing conditions of about 60° C., 0.5×SSC, 0.1% SDS. Conditions of high stringency are as defined by protocol of Chuch and Gilbert 1986, PNAS 81, 1991-1995: Church Buffer: 1 mM EDTA, 0.5 M Na₂HPO₄, pH 7.2, 7% SDS overnight at 65° C. Stringent washing: two times wash in 40 mM Na₂HPO₄, pH 7.2, 1% SDS (washII) at 65° C. The skilled artisan will recognize that the temperature and wash solution salt concentration can be adjusted as necessary according to factors such as the length of the probe.

The Nap1l2 and NAP1L2 polynucleotides of the invention can be utilized to identify target nucleic acids in a pool of nucleic acids. The target nucleic acid can be a nucleic acid (often derived from a biological sample) to which a probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The target nucleic acid may be a specific subsequence (meaning a part of a longer sequence) of a larger nucleic acid (e.g. a fragment of a Nap1l2 gene or of a NAP1L2 gene) to which the probe is directed or to the overall sequence (e.g., a complete Nap1l2 or NAP1L2 gene) whose expression level it is desired to detect.

This invention further provides a method of monitoring the expression (e.g. detecting and/or quantifying the expression) of the Nap1l2 gene or the NAP1L2 gene. The method involves providing a pool of target nucleic acids comprising mRNA transcripts of one or more of these genes, or nucleic acids derived from the mRNA transcripts, hybridizing the pool of nucleic acids to oligonucleotide probes, wherein the oligonucleotide probes are complementary to the mRNA transcripts or nucleic acids derived from the mRNA transcripts, and quantifying the hybridized nucleic acids. The pool of target nucleic acid can be one in which the concentration of the target nucleic acids (mRNA transcripts) or nucleic acids derived from the mRNA transcripts is proportional to the expression levels of the Nap1l2 or NAP1L2 genes. Microfabricated arrays of large numbers of different oligonucleotide probes (e.g. DNA chips) may effectively be used to detect the presence or absence of the target nucleic acid sequences and to quantify the relative abundance of the target sequences in a complex nucleic acid pool.

The pool of target nucleic acids can be the total polyA mRNA isolated from a biological sample, or cDNA made by reverse transcription of the RNA or second strand cDNA or RNA transcribed from the double-stranded cDNA intermediate. The pool of target nucleic acids can be treated to reduce the complexity of the sample and thereby reduce the background signal obtained in hybridization.

The term “quantifying” when used in the context of quantifying transcription levels of a gene can refer to absolute or to relative quantification. Absolute quantification may be accomplished by inclusion of known concentration(s) of one or more target nucleic acids (e.g. control nucleic acids or with known amounts the target nucleic acids themselves) and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

Due to the known degeneracy of the genetic code, wherein more than one codon can encode the same amino acid, a DNA sequence can vary from those described in the examples herein and still encode a mutated Nap1l2 and NAP1L2 polypeptide. Such variant DNA sequences can result from silent mutations (e.g., occurring during PCR amplification), or can be the product of deliberate mutagenesis of a native sequence.

The mutated Nap1l2 and NAP1L2 polynucleotides of the invention are suitable for use in combinatorial strategies for arrays useful in diagnostic applications. For example, an array can be created by moving a dispenser toward a solid support until a tip of the dispenser touches the support, withdrawing the tip from the surface, and releasing a drop of typically 5 nanoliters or less in an area smaller than 1 nm² to create an array of polynucleotides of at least 100 spots. Methods for forming arrays of this type are described in U.S. Pat. No. 6,040,193, the entire disclosure of which is relied upon and incorporated by reference herein.

Arrays utilizing the mutated Nap1l2 and NAP1L2 polynucleotides of the invention can also be prepared by synthesizing oligonucleotides on a solid substrate. For example, one method provides for the irradiation of a first predefined region of the substrate. The irradiation of a second predefined region of the substrate is carried out. The irradiation step removes a protecting group therefrom. The substrate is then contact with a first nucleotide to couple the nucleotide to the substrate in the first predefined region. By repeating these steps, an array of the mutated Nap1l2 and NAP1L2 polynucleotides is formed on the substrate. Suitable methods are described in U.S. Pat. No. 5,424,186, the entire disclosure of which is relied upon and incorporated by reference herein.

These arrays are useful for sequencing, fingerprinting, and mapping mutated Nap1l2 and NAP1L2 polynucleotides of the invention. The methods can make use of a plurality of sequence specific recognition reagents, which can also be used for classification of biological samples and characterization of their sources. An example of a method of identifying nucleotide differences between a native or wild sequence and a mutated Nap1l2 and NAP1L2 polynucleotide in the invention can comprise the steps of providing a substrate having different polynucleotide probes of known sequence at known locations attached at a very high density, contacting a target nucleic acid with the polynucleotide probes attached to the substrate under conditions for high specificity complementary hybridization, determining which polynucleotide probes have hybridized with the target nucleic acid, and using a computer to compare the sequence of the reference nucleic acid with the sequences of the polynucleotide probes that have hybridized with the target nucleic acid and identifying the nucleotide differences between the sequence of the target nucleic acid and the sequence of the reference nucleic acid. Methods of this type are disclosed in detail in U.S. Pat. No. 5,925,525, the entire disclosure of which is relied upon and incorporated by reference herein. A suitable computer is described in U.S. Pat. No. 5,974,164.

The polynucleotides employed in the invention can be labeled before, during, or after hybridization. Fluorescent labels are particularly preferred, and where used, quantification of the hybridized polynucleotides is by quantification of fluorescence from the hybridized fluorescently labeled nucleic acids.

The arrays can also be utilized for expression monitoring by hybridization to the high density oligonucleotides. For example, a typical method of analyzing the expression of one or more polynucleotides of the invention comprises providing a pool of target nucleic acids comprising RNA transcripts of one or more of the genes, or nucleic acids derived therefrom using RNA transcripts as templates, and hybridizing the pool of target nucleic acids to an array of nucleotide probes immobilized on the surface. One such array described in U.S. Pat. No. 6,040,138 comprises more than one 100 different oligonucleotides, at least some of which comprise control probes. Each different oligonucleotide is localized in a predetermined region on the surface. The density of the different oligonucleotides is greater than about 60 different oligonucleotides per 1 cm² . In addition, at least some of the oligonucleotide probes are complementary to RNA transcripts or the nucleic acids derived therefrom using the RNA transcripts. Hybridization of the nucleic acids to the array can be quantified such that quantification is proportional to the expression level of the genes.

Recombinant expression vectors containing a nucleic acid sequence encoding mutated Nap1l2 and NAP1L2 polypeptides can be prepared using well known methods. The expression vectors include a mutated Nap1l2 and NAP1L2 DNA sequence operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences, which control transcription and translation initiation and termination. Nucleotide sequences are “operably linked” when the regulatory sequence functionally relates to the mutated Nap1l2 and NAP1L2 DNA sequence. Thus, a promoter nucleotide sequence is operably linked to a mutated Nap1l2 or NAP1L2 DNA sequence if the promoter nucleotide sequence controls the transcription of the mutated Nap1l2 or NAP1L2 DNA sequence. The ability to replicate in the desired host cells, usually conferred by an origin of replication, and a selection gene by which transformants are identified can additionally be incorporated into the expression vector.

In addition, sequences encoding appropriate signal peptides that are not naturally associated with mutated Nap1l2 and Nap1l2 polypeptides can be incorporated into expression vectors. For example, a DNA sequence for a signal peptide (secretory leader) can be fused in-frame to the mutated Nap1l2 and NAP1L2 polynucleotide sequence so that the mutated Nap1l2 and NAP1L2 polypeptide is initially translated as a fusion protein comprising the signal peptide. A signal peptide that is functional in the intended host cells enhances extracellular secretion of the mutated Nap1l2 and NAP1L2 polypeptide. The signal peptide can be cleaved from the mutated Nap1l2 and NAP1L2 polypeptide upon secretion of mutated Nap1l2 and NAP1L2 polypeptide from the cell.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids. Commercially available vectors include those that are specifically designed for the expression of proteins. These include pMAL-p2 and pMAL-c2 vectors, which are used for the expression of proteins fused to maltose binding protein (New England Biolabs, Beverly, Mass., USA). Examples of other expression vectors, which can be used in the present invention, are

Bacteria:

pQE vector system (Qiagen); and

pCAL vector system (Stratagene);

Mammalian:

example for inducible system: pTet-ON & pTet-OFF (Clontech);

example for constitutive expression: pCMV vectors (±Tag) (Stratagene);

Baculo virus Expression System (e.g. Clontech, Stratagene, Invitrogen);

Yeast Expression vectors (e.g. Clontech, Stratagene, Invitrogen); and

In vitro Expression pSPUTK vector (Stratagene).

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776), and tac promoter (Maniatis, Molecular Cloning: A Laboratory Manual, Cold Spring Harbor Laboratory, p. 412, 1982). Inducible promoters belong to these systems.

A substantial part of this invention lies in the characterization of the promoter sequence of the Nap1l2 and NAP1L2 genes. Especially due to the specific expression of NAP1L2, which has been discovered by the present invention, this promoter can be used in the construction of vectors or polynucleotides useful for expression or the modulation of the expression of endogenous or exogenous genes or fragments of genes of interest corresponding to a biological product produced in vivo or in vitro in neural cells, preferably in post-mitotic neural cells. Either endogenous or exogenous genes can be targeted and expressed in such cells, for example, for therapeutic purposes.

The genomic sequences of the Nap1l2 and NAP1L2 genes and their promoters are shown in FIGS. 7-10. The promoter sequence for the mouse Nap1l2 gene is included in the sequence (i.e., it is a subsequence of the sequence) entitled Sequence clone Nap1l2 promoter murin SpeI-SalI (FIG. 7). The sequence entitled Human NAP1L2 5′ region (FIG. 10) contains (as a subsequence) the promoter for the human NAP1L2 gene.

Suitable host cells for expression of mutated Nap1l2 and NAP1L2 polypeptides include prokaryotes, yeast, or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, N.Y., (1985). Cell-free translation systems could also be employed to produce mutated Nap1l2 and NAP1L2 polypeptides using RNAs derived from DNA constructs disclosed herein.

It will be understood that the present invention is intended to encompass the previously described mutated proteins in isolated or purified form, whether obtained using the techniques described herein or other methods. In a preferred embodiment of this invention, the mutated Nap1l2 and NAP1L2 polypeptides are substantially free of human tissue and human tissue components, nucleic acids, extraneous proteins and lipids, and adventitious microorganisms, such as bacteria and viruses. It will also be understood that the invention encompasses equivalent mutated proteins having substantially the same biological and immunogenic properties. Thus, this invention is intended to cover serotypic variants of the mutated proteins of the invention.

Depending on the use to be made of the mutated Nap1l2 and NAP1L2 polypeptides of the invention, it may be desirable to label them. Examples of suitable labels are radioactive labels, enzymatic labels, fluorescent labels, chemiluminescent labels, and chromophores. The methods for labeling mutated proteins and glycoproteins of the invention do not differ in essence from those widely used for labeling immunoglobulin. The need to label may be avoided by using labeled antibody to the antigen of the invention or anti-immunoglobulin to the antibodies to the antigen as an indirect marker. The mutated proteins can be detected by expression of easily detectable proteins such as LacZ or GFP.

Once the mutated Nap1l2 and NAP1L2 polypeptides of the invention have been obtained, they can be used to produce polyclonal and monoclonal antibodies reactive therewith. Thus, a mutated protein or polypeptide of the invention can be used to immunize an animal host by techniques known in the art. Such techniques usually involve inoculation, but they may involve other modes of administration. A sufficient amount of the mutated protein or the polypeptide is administered to create an immunogenic response in the animal host. Any host that produces antibodies to the antigen of the invention can be used. Once the animal has been immunized and sufficient time has passed for it to begin producing antibodies to the antigen, polyclonal antibodies can be recovered. The general method comprises removing blood from the animal and separating the serum from the blood. The serum, which contains antibodies to the antigen, can be used as an antiserum to the antigen. Alternatively, the antibodies can be recovered from the serum. Affinity purification is a preferred technique for recovering purified polyclonal antibodies to the antigen, from the serum.

Monoclonal antibodies to the antigens of the invention can also be prepared. One method for producing monoclonal antibodies reactive with the antigens comprises the steps of immunizing a host with the antigen; recovering antibody producing cells from the spleen of the host; fusing the antibody producing cells with myeloma cells deficient in the enzyme hypoxanthine-guanine phosphoribosyl transferase to form hybridomas; select at least one of the hybridomas by growth in a medium comprising hypoxanthine, aminopterin, and thymidine; identifying at least one of the hybridomas that produces an antibody to the antigen, culturing the identified hybridoma to produce antibody in a recoverable quantity; and recovering the antibodies produced by the cultured hybridoma.

These polyclonal or monoclonal antibodies can be used in a variety of applications. Among these is the neutralization of corresponding mutated proteins. They can also be used to detect antigens in biological preparations or in purifying corresponding mutated proteins, glycoproteins, or mixtures thereof, for example when used in a affinity chromatographic columns.

The mutated Nap1l2 and NAP1L2 polypeptides can be used as antigens to identify antibodies in materials and to determine the concentration of the antibodies in those materials. Such materials of course include human and other animal tissue and human and other animal cells, as well as biological fluids, such as human and other animal body fluids, including human and other animal sera. When used as a reagent in an immunoassay for determining the presence or concentration of the antibodies, the antigens of the present invention provide an assay that is convenient, rapid, sensitive, and specific.

More particularly, the antigens of the invention can be employed in immunoassays that are well known for use in detecting or quantifying humoral components in fluids. Thus, antigen-antibody interactions can be directly observed or determined by secondary reactions, such as precipitation or agglutination. In addition, immunoelectrophoresis techniques can also be employed. For example, the classic combination of electrophoresis in agar followed by reaction with anti-serum can be utilized, as well as two-dimensional electrophoresis, rocket electrophoresis, and immunolabeling of polyacrylamide gel patterns (Western Blot or immunoblot.) Other immunoassays in which the antigens of the present invention can be employed include, but are not limited to, radioimmunoassay, competitive immunoprecipitation assay, enzyme immunoassay, and immunofluorescence assay. It will be understood that turbidimetric, colorimetric, and nephelometric techniques can be employed. An immunoassay based on Western Blot technique is preferred.

Immunoassays can be carried out by immobilizing one of the immunoreagents, either an antigen of the invention or an antibody of the invention to the antigen, on a carrier surface while retaining immunoreactivity of the reagent. The reciprocal immunoreagent can be unlabeled or labeled in such a manner that immunoreactivity is also retained. These techniques are especially suitable for use in enzyme immunoassays, such as enzyme linked immunosorbent assay (ELISA) and competitive inhibition enzyme immunoassay (CIEIA).

When either the antigen of the invention or antibody to the antigen is attached to a solid support, the support is usually a glass or plastic material. Plastic materials molded in the form of plates, tubes, beads, or disks are preferred. Examples of suitable plastic materials are polystyrene and polyvinyl chloride. If the immunoreagent does not readily bind to the solid support, a carrier material can be interposed between the reagent and the support. Examples of suitable carrier materials are proteins, such as bovine serum albumin, or chemical reagents, such as gluteraldehyde or urea. Coating of the solid phase can be carried out using conventional techniques.

The term “antibodies” is meant to include polyclonal antibodies, monoclonal antibodies, fragments thereof, such as F(ab′)2 and Fab fragments, as well as any recombinantly produced binding partners. Antibodies are defined to be specifically binding if they bind mutant Nap1l2 and NAP1L2 polypeptides with a Kₐ of greater than or equal to about 10⁷ M⁻¹. Affinities of binding partners or antibodies can be readily determined using conventional techniques, for example, those described by Scatchard et al., Ann. N.Y Acad. Sci., 51:660 (1949). Polyclonal antibodies can be readily generated from a variety of sources, for example, horses, cows, goats, sheep, dogs, chickens, rabbits, mice, or rats, using procedures that are well known in the art.

To further achieve the objects and in accordance with the purposes of the present invention, a kit is provided. This kit, in one embodiment, contains the DNA sequences of this invention, which are capable of hybridizing to RNA or analogous DNA sequences to indicate the presence of a mutated polynucleotide of the invention. Different diagnostic techniques can be used which include, but are not limited to: (1) Southern blot procedures to identify cellular DNA which may or may not be digested with restriction enzymes; (2) Northern blot techniques to identify RNA extracted from cells; and (3) dot blot techniques, i.e., direct filtration of the sample through an ad hoc membrane, such as nitrocellulose or nylon, without previous separation on agarose gel. Suitable material for dot blot technique could be obtained from body fluids including, but not limited to, serum and plasma, supernatants from culture cells, or cytoplasmic extracts obtained after cell lysis and removal of membranes and nuclei of the cells by centrifugation.

Another aspect of the present invention is directed to a transgenic animal carrying a polynucleotide according to the invention. According to a preferred embodiment of the invention, the animal is a mammal, and more preferably a mouse. The transgenic animal according to the invention can be produced by different techniques available to the skilled man in the art, in order for example to study the function of the mutated Nap1l2 and NAP1L2 genes in mammals. Mouse lines in which the Nap1l2 gene or the NAP1L2 gene have been mutated can be generated.

Gene targeting techniques can be used to introduce therapeutic polynucleotides, e.g. naturally occurring unmutated, Nap1l2 and NAP1L2 genes, into a host cell containing a mutated Nap1l2 or NAP1L2 gene. One of the preferred targeting techniques according to the present invention consists of a process for specific replacement, such as the DNA targeting technique described in PCT patent application No. WO 90/11354 (Institut Pasteur), incorporated herein by reference. Such a DNA targeting process makes it possible to insert the therapeutic nucleotide according to the invention behind an endogenous promoter, which has the desired functions (for example, specificity of expression in the selected target animal or embryo).

Absence of NAP1L2 protein (especially due to mutations of the corresponding genes or of their promoters) leads to overproduction of neural cells; expression of NAP1L2 or subfragments or derivatives in cells (neural/tumors/others) can prevent further proliferation and then can be used as a therapy. On the contrary, modification of Nap1L2/NAP1L2 expression (especially due to mutations of these genes or inefficiency of their promoters) leads to over production of neural cells and thereby allow regeneration or survival of neurons and therefore use as a therapy.

The following plasmids were deposited at the Collection National de Cultures de Microorganismes (C.N.C.M.), of Institut Pasteur, 28 rue due Docteur Roux, F-75724 Paris, Cedex 15, France, and assigned the following Accession Nos.:

| PLASMID | DEPOSIT DATE | ACCESSION NO. |
|---|---|---|
| pCUR1-2 | April 25, 2000 | I-2463 |
| BPX-1 | April 25, 2000 | I-2464 |
| BPX-2 | April 25, 2000 | I-2465 |
| BPX-3 | April 25, 2000 | I-2466. |

Another aspect of the invention is an eukaryotic cell containing the insert contained in the plasmid BPX-1 or BPX-2 or BPX-3 or polynucleotides hybridizing under stringent conditions with the said insert.

This invention will now be described in greater detail in the following Examples.

### EXAMPLE 1

### Expression Profile Analysis During Mouse Development

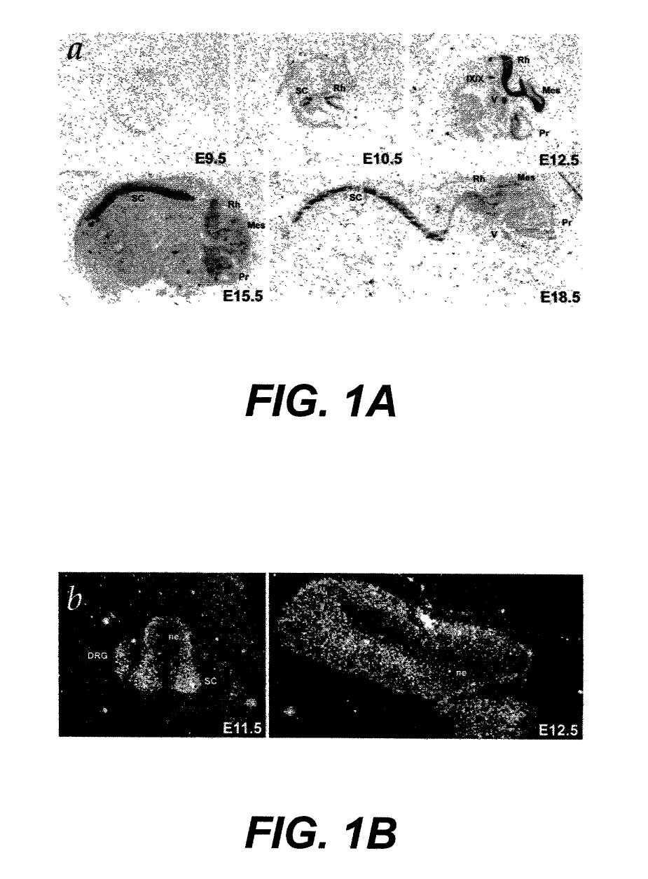

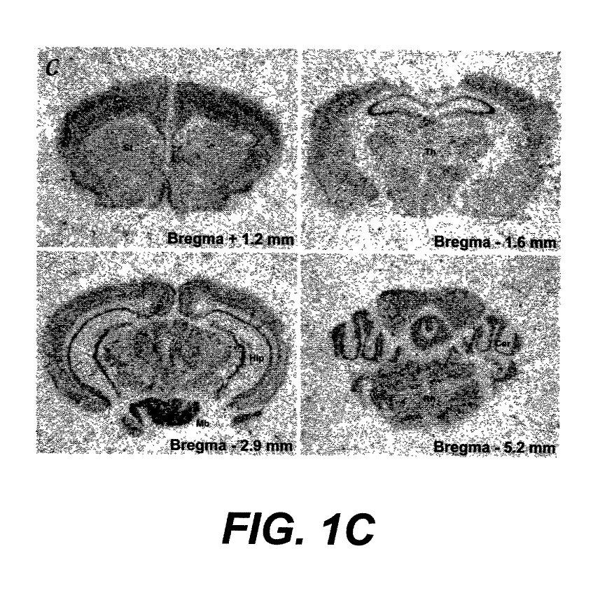

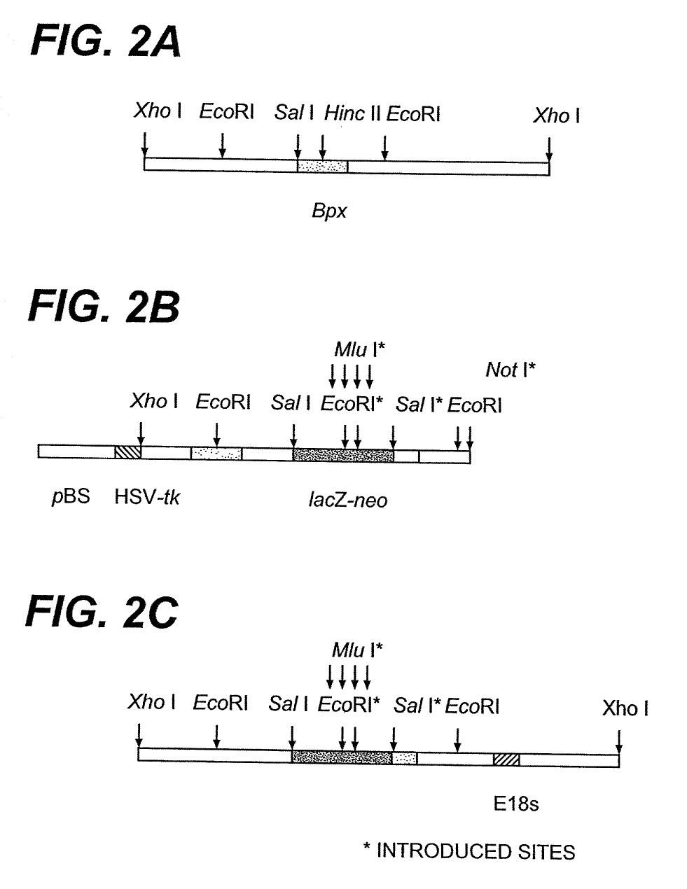

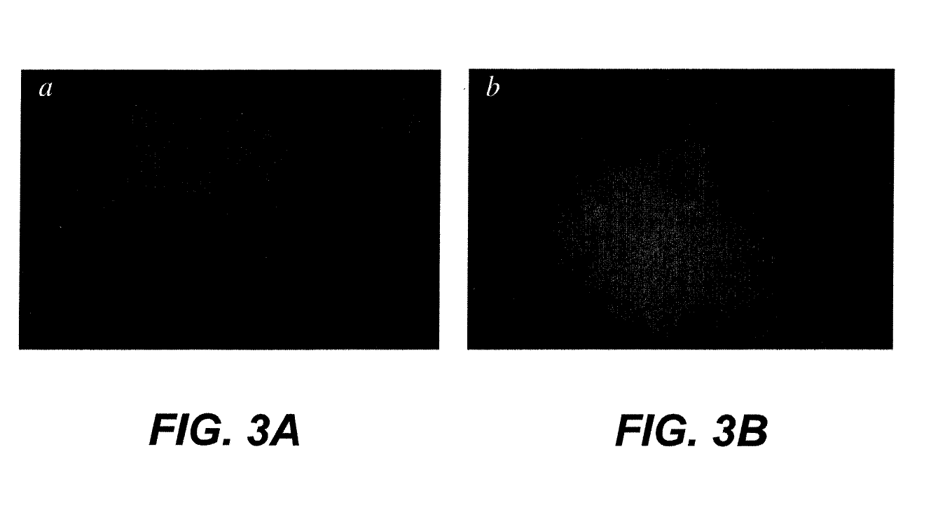

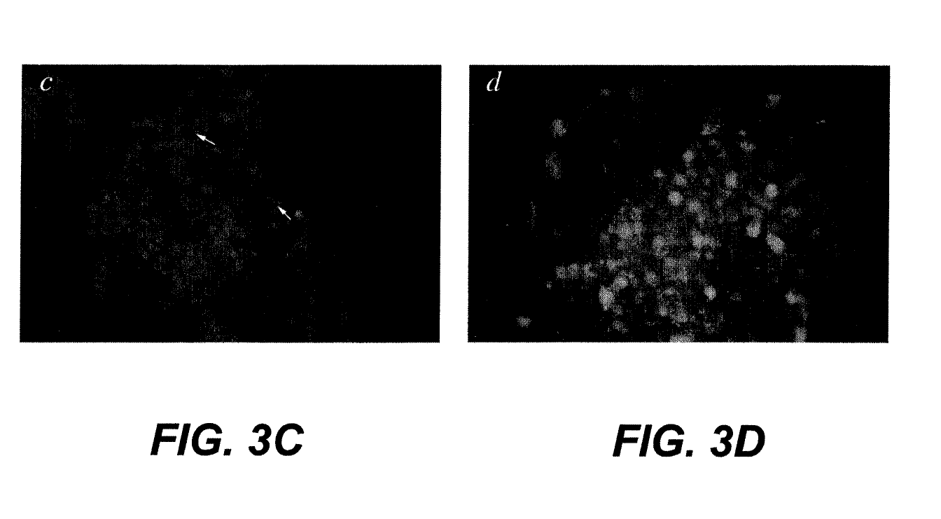

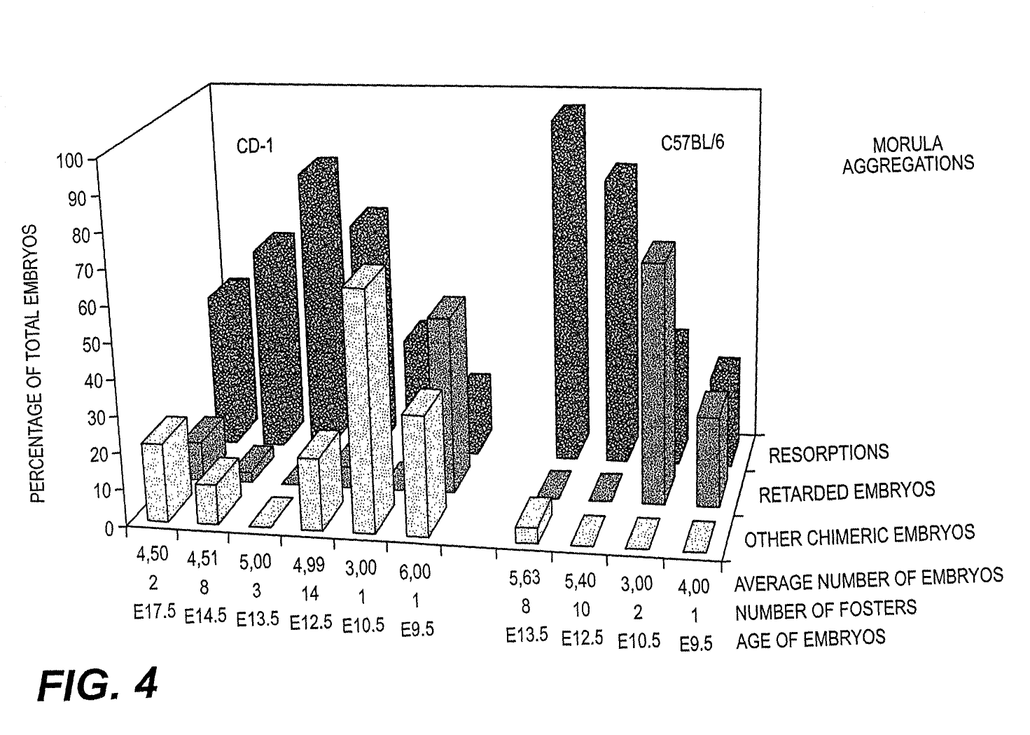

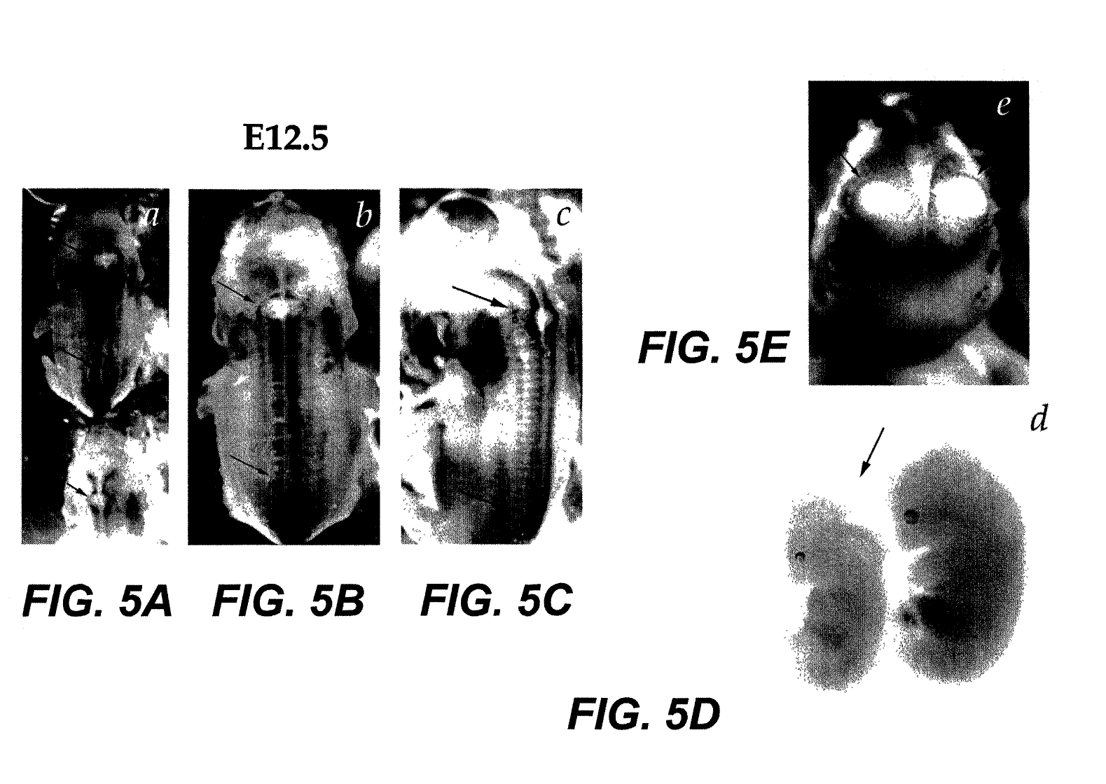

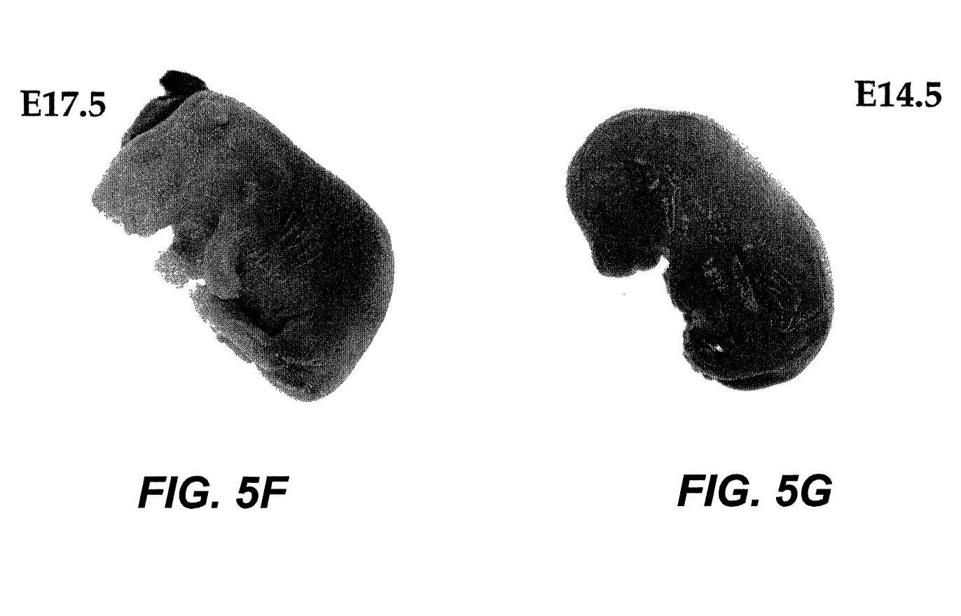

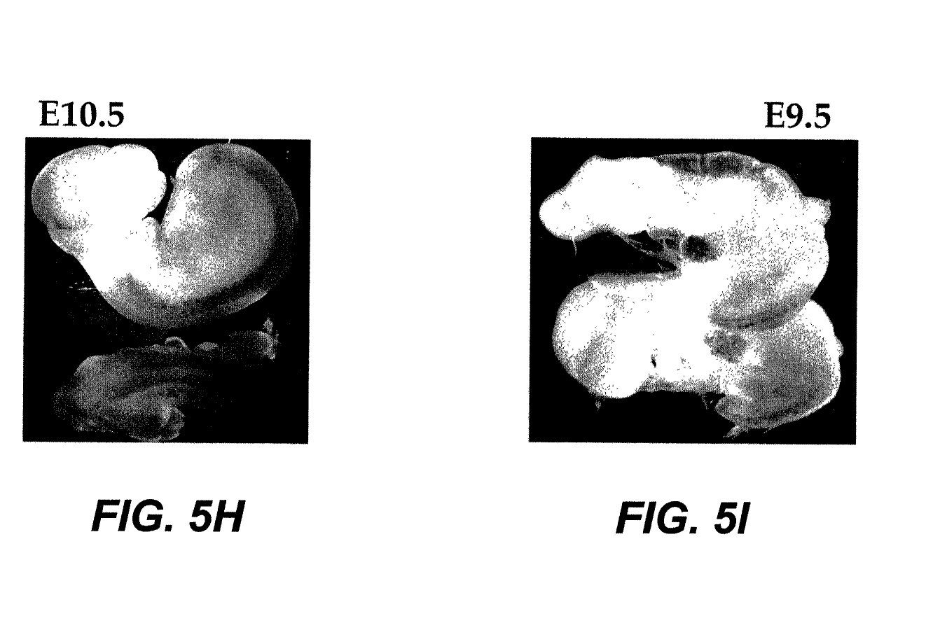

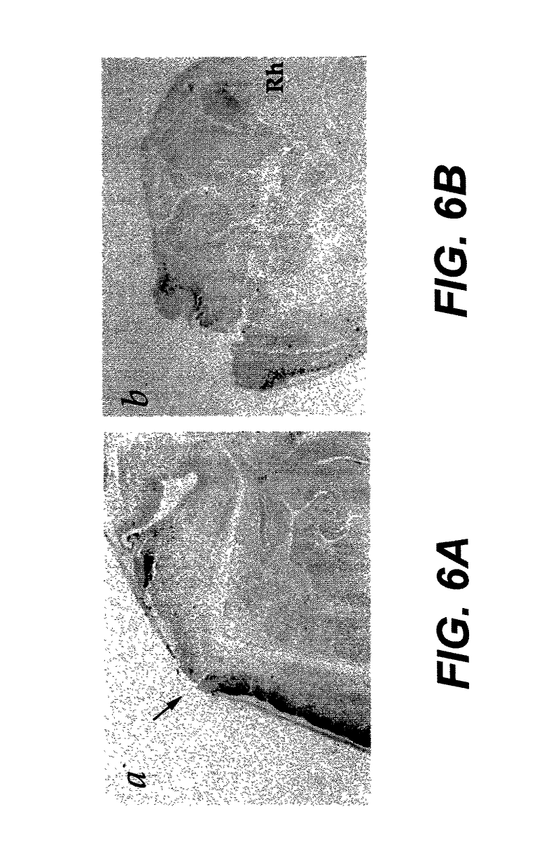

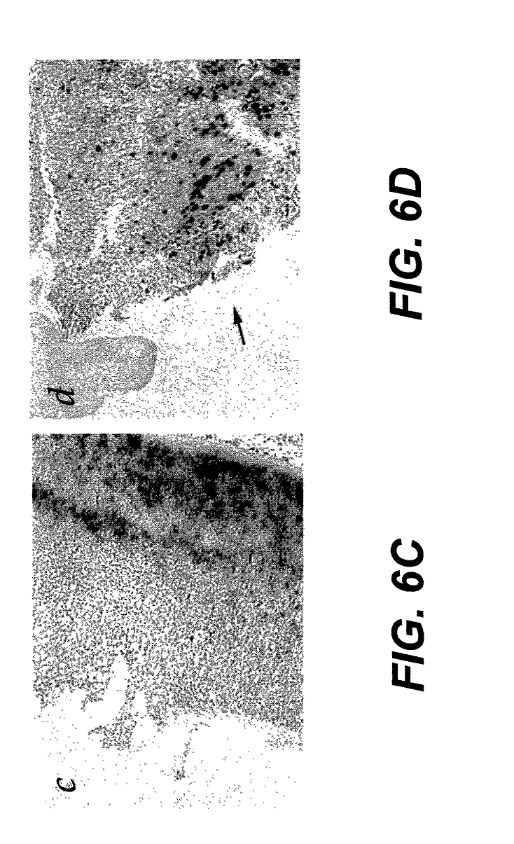

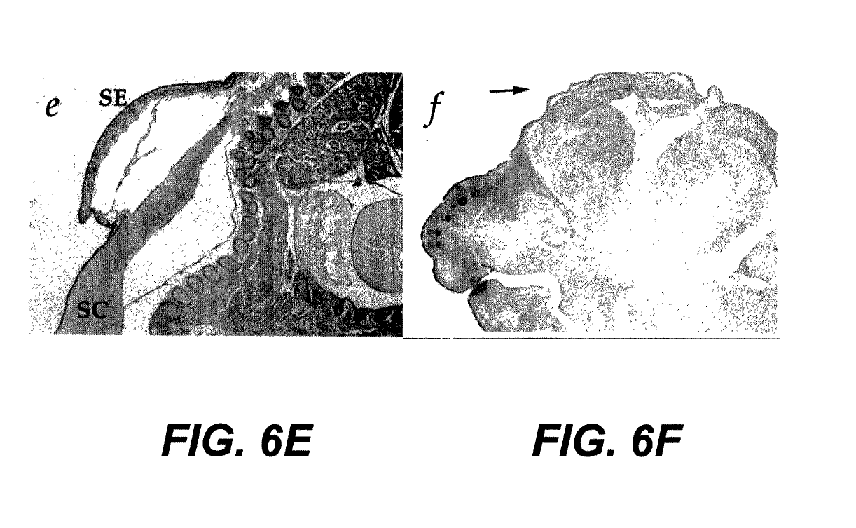

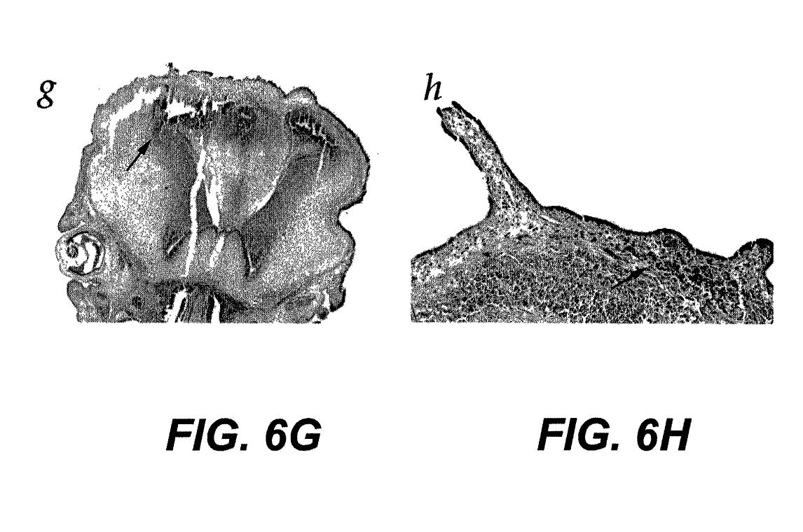

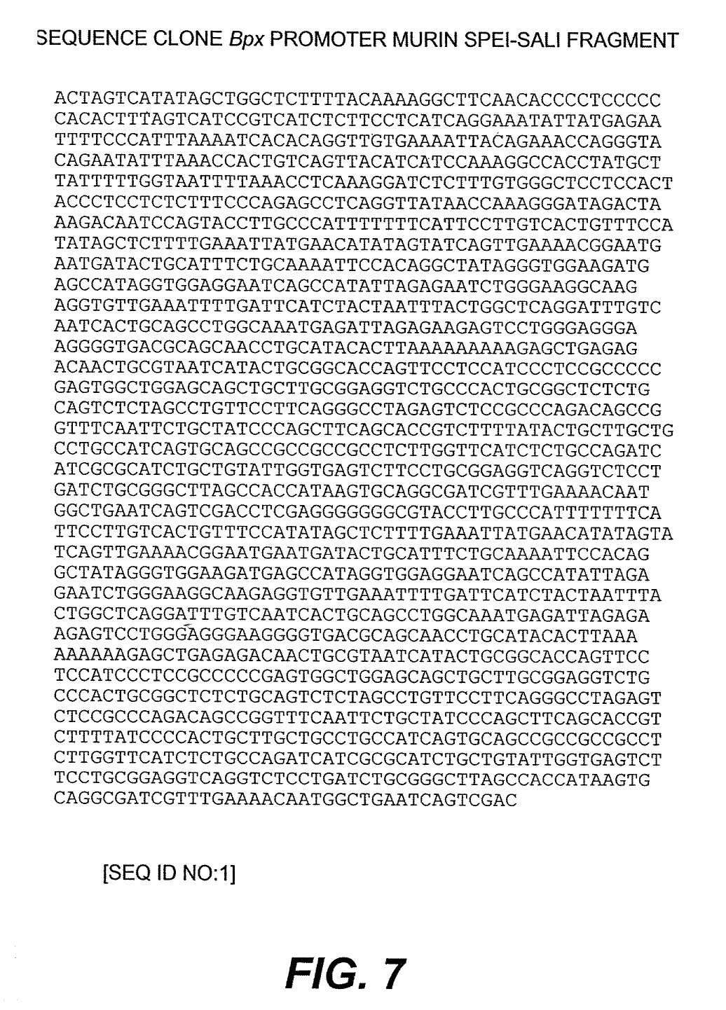

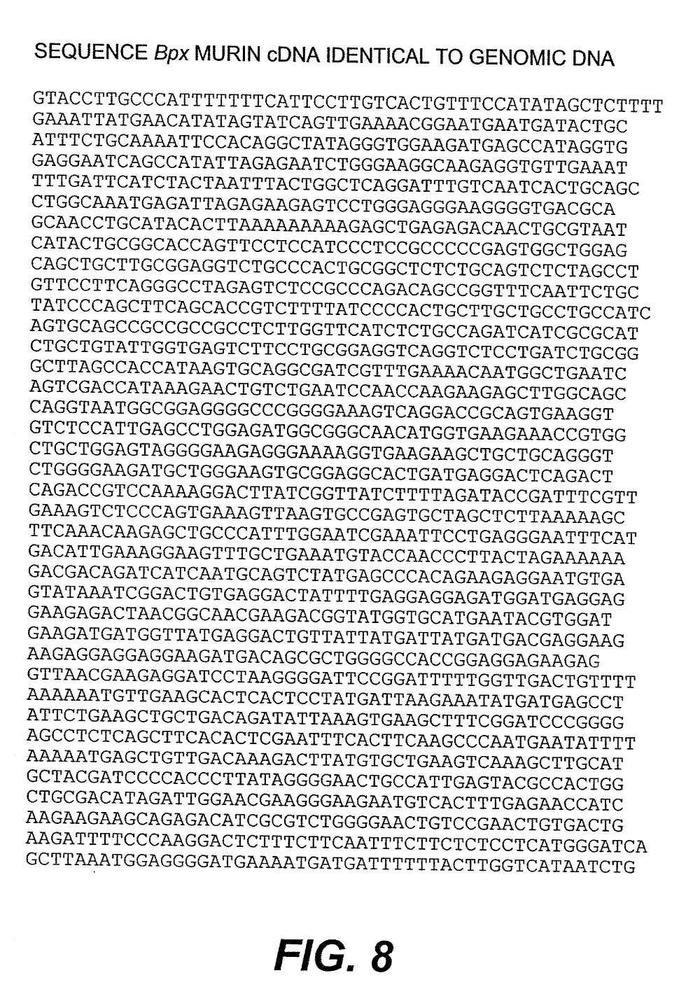

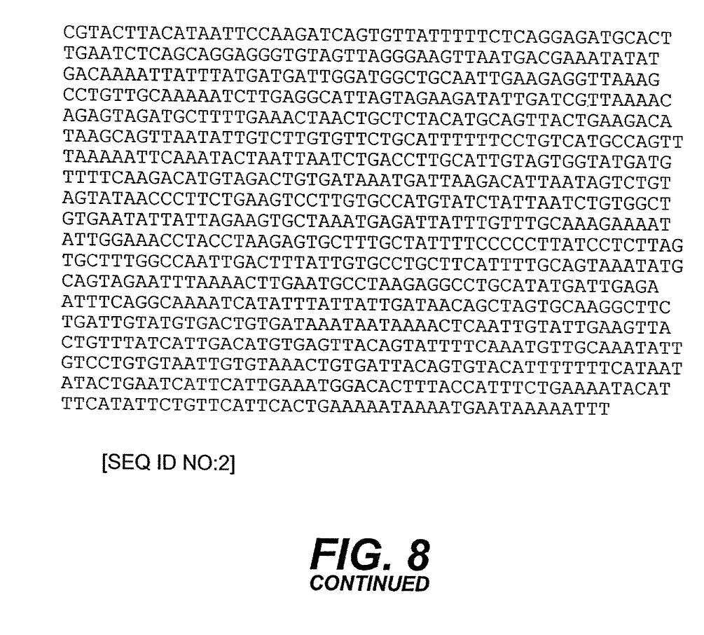

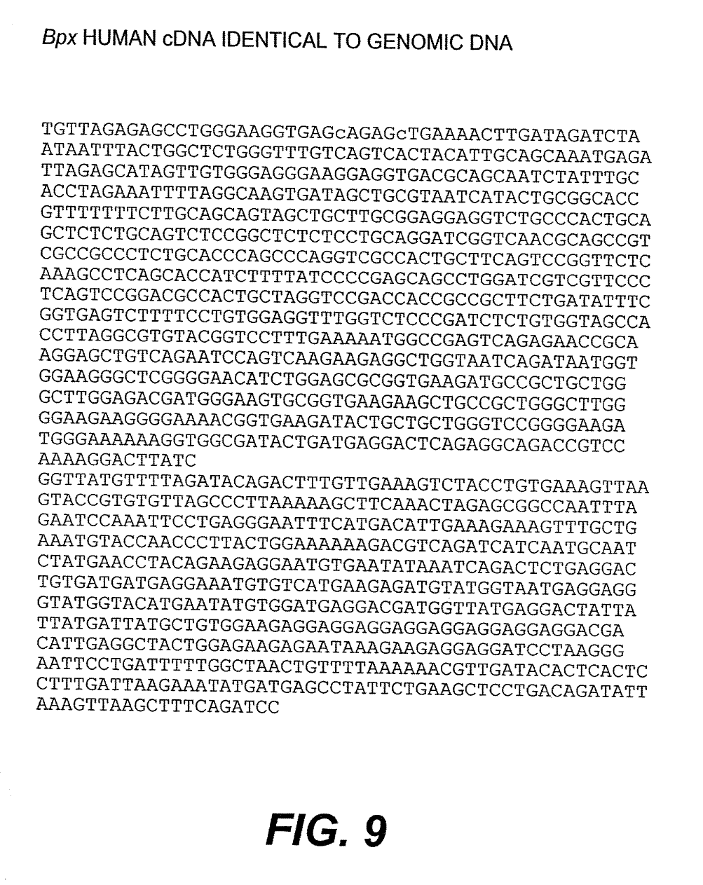

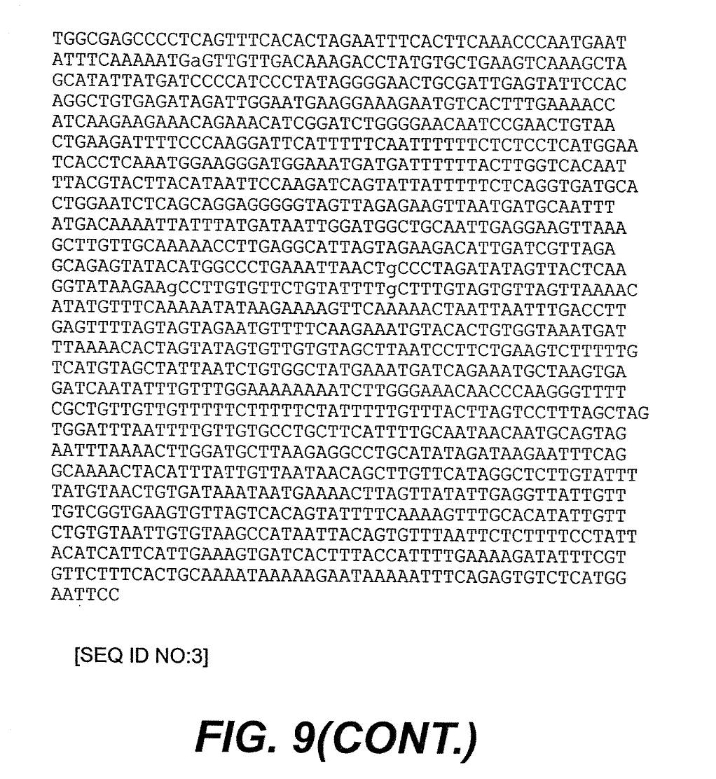

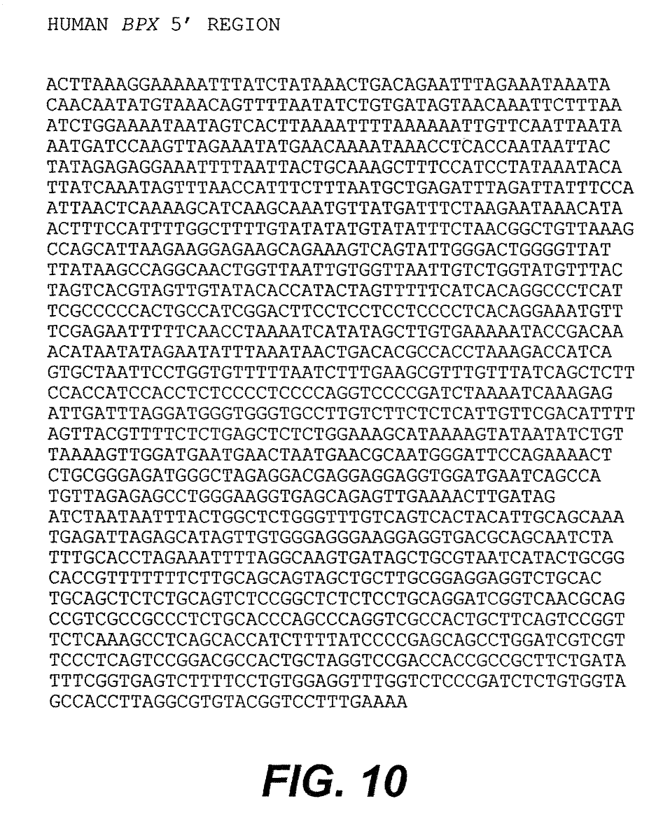

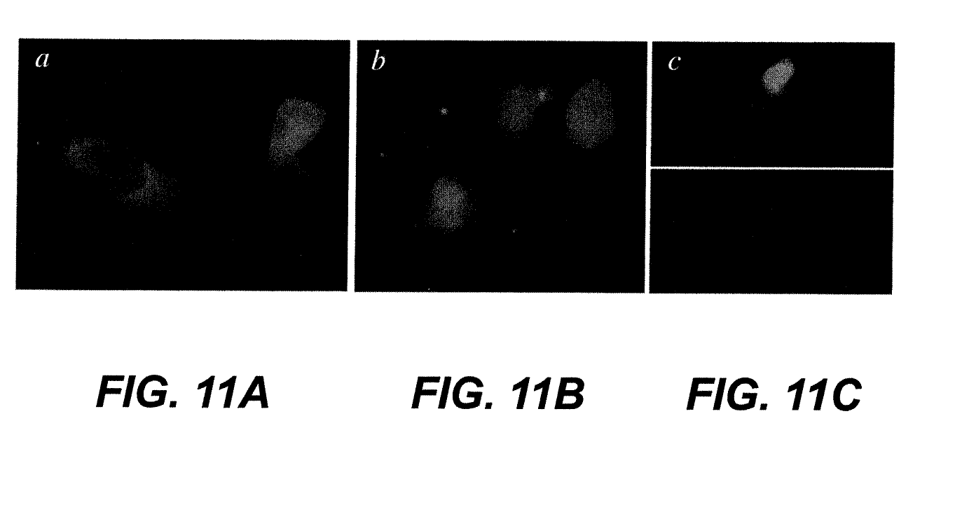

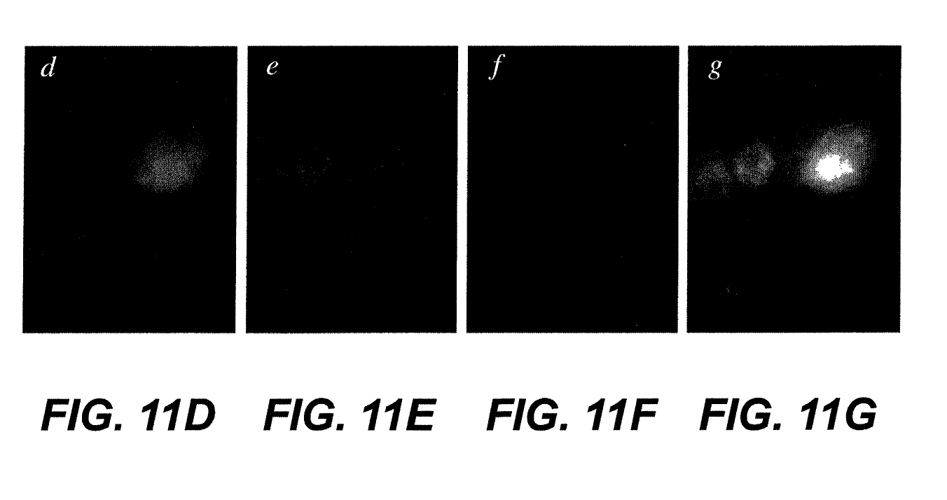

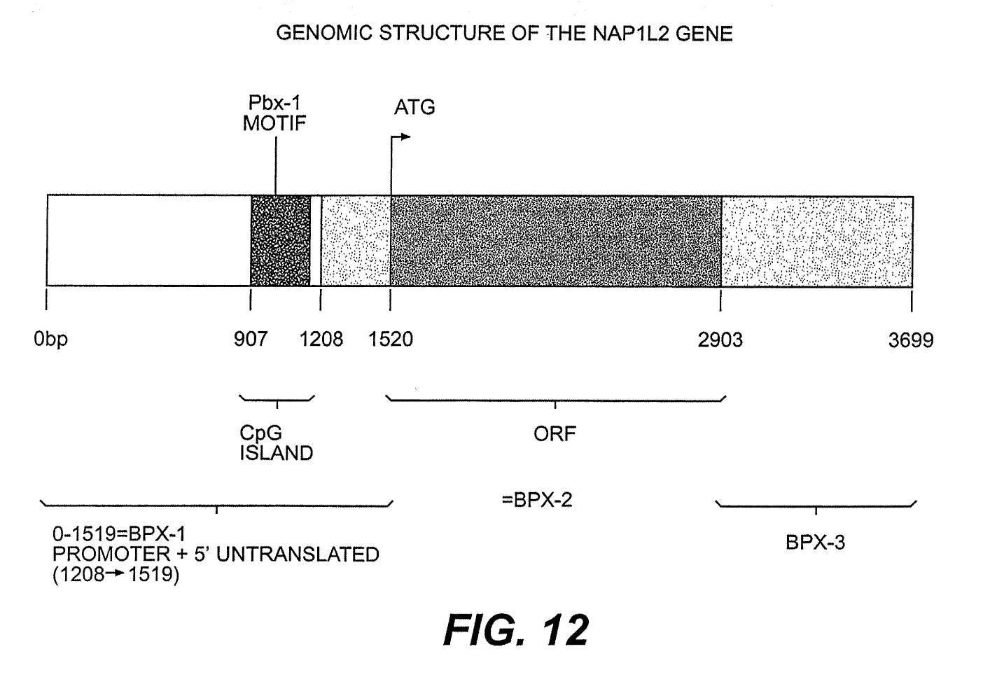

In order to obtain a more precise overview of the profile of Nap1l2 expression, RNA in situ hybridization was performed using a Nap1l2 specific oligonucleotide probe on sections of adult mouse brain and mouse embryos corresponding to days E5.5 through to E18.5 (FIG. 1).

Nap1l2 expression was found throughout the nervous system, in structures belonging to both the central and peripheral nervous systems. Expression was first detectable at day E10.5, and correlates with the initial wave of neuronal differentiation (FIG. 1). Embryonic stages E10.5 through to E18.5 revealed that Nap1l2 expression, although predominantly in the spinal cord, was also present throughout the brain and ganglia. In the adult brain, all regions were labeled, although variation in the intensity of the labeling suggested some heterogeneity in expression levels. Signals were particularly strong in the anterior olfactory nucleus, the hippocampus, the hypothalamus and the cerebellum. The strongest Nap1l2 signal was detected in the mammillary bodies (see Bregma −2.9, FIG. 1).

Differentiated regions within the nervous system exhibited strong labeling, whereas ventricular zones did not show specific signals. No Nap1l2 transcripts could be detected in glial cells or in tissues other than the nervous system. Expression of Nap1l2 is likely to be restricted to post-mitotic neurons.

### EXAMPLE 2

### The Role of Nap1l2 Defined by Deletion Analysis Targeted Deletion and Differentiation of ES Cells

In order to establish the role of Nap1l2 we created a null mutation of the Nap1l2 gene in male ES cells, hemizygous for Nap1l2, by homologous recombination. In the knockout construct, the intronless Nap1l2 gene was partially deleted and replaced by a β-galactosidase reporter and neomycin resistance gene (FIG. 2). The resulting fusion protein has potential for Nap1l2 function, since it includes only five amino acids from the N-terminal end of NAP1L2, all the non-deleted C-terminal sequences being out of frame. Two targeted cell lines, 5b17 and 8b21, in which the endogenous X-linked Nap1l2 gene had been replaced by homologous recombination, were obtained (FIG. 2). The absence of a Nap1l2 transcript in these ES clones was confirmed by RT-PCR, and the karyotype of the clones verified on mitotic spreads.

As ES cells have the potential to differentiate into neurons in vitro, an investigation was made to determine whether the deletion of Nap1l2 affects the in vitro development of neurons. In vitro differentiation experiments are based on the formation of embryoid bodies in suspension culture. Re-attachment of the embryoid bodies after four days of culture leads to the formation of various types of differentiated cells. Formation of neurons can be induced by the addition of retinoic acid to the medium¹⁸. To visualize the specific cell types formed, antibodies directed against various neuronal marker proteins: nestin, which is present in precursor cells, β-tubulin III in early neurons, NF200 in differentiated neurons, and GFAP in glial cells, were used.

All three cell lines, the original ES cell line CK35 and the two recombinant ES cell lines, 5b17 and 8b21, were able to form neurons the presence of retinoic acid. In both the normal and mutant cell lies, nestin positive cells were observed two or three days after attachment of the embryoid bodies. Neurons together with glial cells usually appeared four to six days after embryoid body replating. The number of neurons developing was dependent on the concentration of retinoic acid used¹⁹. Whereas cultures without retinoic acid produced only a few neuronil cells, their number was substantially increased by adding 3×10⁻³ M retinoic acid.

In the absence of retinoic acid, the CK35 cell line produced as expected, only few neuronal cells. In contrast, the mutant cell lines produced large numbers of nestin positive neuronal cells increasing from about 50 cells per mm² one day to 200 cells per mm² three days after re-attachment (FIGS. 3a, b). Many of these nestin positive cells were lacZ positive (FIG. 3c). Pulse chase experiments using BrdU confirmed that these lacZ expressing cells represent a growing cell population (data not shown).

These experiments show that the Nap1l2 mutation affects the proliferation of neuronal precursor cells in vivo as well as in vitro. The dual effect of RA on both neuronal cell differentiation and G1 arrest of cell division¹³ probably leads to the suppression of the proliferative effect of the Nap1l2 mutation.

### EXAMPLE 3

### Phenotypical Observation of Chimeras

In order to examine more closely the effect of the Nap1l2 deletion on mouse development, changes in the nervous system in vivo were examined. Chimeric embryos were obtained by blastocyst injection and morula aggregation using both mutant ES cell lines.

A total of 153 chimeric blastocysts were generated by blastocyst injection using the 8b21 ES cell line. Of the 34 pups born, only three were chimeric as judged by coat color. Two of these were less than 20% chimeric. The only high percentage chimera (80%) was a weak runt and died at one week post-partum. Similar results were obtained with the 5b17 line. In no case was germline transmission found. In our hands, blastocyst injections generally give higher birth rates, 25 to 50% chimerism and germline transmission in over 40% of the resulting chimeras. The results of these Nap1l2 knockout ES cell chimeras indicated possible prenatal lethality. This was not unlikely, considering that the targeted ES cells are male and the Nap1l2 gene X-linked.

ES cell morula aggregates were also generated with both ES cell lines. Foster mothers were implanted with 8-12 morula aggregates using either CD-1 (albino) or C57BL/6 morulas. Very similar results in these experiments to that observed with the blastocyst injections were obtained. In 12 CD-1 morula aggregation experiments, a total of 36 live births, eight dead pups, and six resorptions were obtained. Judged by coat color, all 36 live births were low percentage chimeras with less than 10% chimerism. On the other hand, of the eight dead newborns, six were highly chimeric. In four experiments using C57BL/6 morulas, one low percentage chimera, one dead pup, and 12 resorptions were obtained.

Whilst low percentage chimerism is compatible with viability, high levels of chimerism result in prenatal lethality. No germline transmission was observed when the low percentage chimeras were mated.

Given the very low birth rates and the low percentage of chimerism, we examined embryos at different time points after reimplantation were studied. Foster mothers were sacrificed at days E9.5, 10.5, 12.5, 13.5, 14.5, and 17.5, and embryos stained for lacZ expression. Both of the two independent targeted ES cell lines, 5b17 and 8b21, again gave similar results (FIG. 4).

A particularly large number of resorptions was observed between days E12.5 and E14.5, whilst retarded embryos were mainly observed between days E9.5 to E10.5. It is likely that these retarded embryos represent the resorptions observed at later embryonic stages (FIG. 4).

In general, significantly less embryos were recovered from the C57BL/6 morula aggregation experiments (FIG. 4), suggesting that genetic background may be an important factor in determining the severity of the phenotype.

NLS-lacZ expression was first detected in normally developed embryos in the caudal tip of the tail at day E9.5 (FIG. 5) and extended to the entire neural tube by day E10.5.

A highly reproducible phenotype was observed in surviving day E12.5 embryos. Chimeric embryos had defects in the surface ectoderm, and were strikingly characterized by failure of neural tube closure, typically in the upper and lower thoracic region (FIG. 5, E12.5, a, b, and c). The percentage of chimerism correlated well with the severity of the neural tube defect (compare a versus b). The most marked phenotype was, however, found in chimeric embryos, which exhibited strong hindbrain ablation or exencephaly (FIG. 5 E12.5d). In the E12.5 embryo shown in FIG. 5e, the surface ectoderm failed cover the telencephalon. A similar phenotype was found in chimeric embryos surviving until day E17.5, which had exposed brains or anencephaly (FIG. 5).

Embryos surviving to mid- or late gestation showed an additional set of phenotypes. Surviving embryos included two chimeric E13.5 embryos (C57BL/6 morula aggregates) which superficially appeared to be phenotypically normal, but which showed strong lacZ-staining over the entire surface ectoderm that correlated with its overdevelopment as seen on tissue sections (see below). One well developed chimeric embryo recovered at day E14.5 had the skin on its back detached from the rest of the body beginning laterally along the trunk and extending dorsally, although remained attached to the body along the dorsal midline (FIG. 5).

### EXAMPLE 4

### Analysis of Sections of Chimeric Embryos

To understand the structural changes underlying the phenotypes observed in chimeric embryos, sections of embryonic tissue were analyzed. Embryos in toto or sections of frozen embryos were stained for lacZ expression. In all cases, the sections were counterstained with hematoxylin or toluidine blue to visualize the different tissues.

Whilst surviving embryos at days E9.5 and E10.5 (FIG. 5) appeared normal, at day E12.5, a clear, reproducible mutant phenotype was observed, most notably in highly chimeric embryos.

Strong lacZ expression, found mainly in the rhombencephalon and in the spinal cord, correlated with the failure of neural tube closure at this position (FIGS. 6, a and c). The failure of neural tube closure was also found in the lower thoracic region of some embryos. In these embryos, the neural tube was exposed and not covered by surface ectoderm. Often, the surface ectoderm and the spinal cord were detached from the rest of the body (FIGS. 6, a and e).

At day E12.5 lacZ staining was also found in the brain, but this staining was generally fainter than in the spinal cord, in agreement with the in situ results (FIG. 1). Major rearrangements of the brain were observed. Large parts of the brain, including the telencephalon, diencephalon, and mesencephalon (FIG. 6b), were greatly expanded as a result of a marked increase in neuronal cells. The resulting neural tissues were disorganized, and the normal separation of neuroepithelium and neurons barely detectable.

Similar rearrangements of the brain were found in chimeric embryos recovered at day E13.5. In addition, a remarkable overdevelopment of the surface ectoderm was found at this stage (FIGS. 6, f and h). Surprisingly, this did not correlate with expression of lacZ in the skin, but in the mesenchyme. Nap1l2 transcripts had not previously been detected by in situ hybridization in this tissue. This defect in the surface ectoderm was also noted in day E14.5 chimeras. Interestingly, by this stage, the regions of the brain that showed overdevelopment at day E12.5 appeared to have become necrotic (FIG. 6g).

Embryos surviving to day E17.5 that exhibited severe anencephaly (FIG. 5), had the roof of the dorsal part of the brain entirely missing, exposing the remaining parts of the brain.

Taken together, these results suggest that the absence of Nap1l2 function leads to an overproduction of cells in the neural tube and the surface ectoderm, and that this interferes with the proper histogenesis of these tissues.

To understand the function of the NAP1L2 protein in proliferating cells, we studied its localization in P19 embryonal carcinoma cells (EC)¹⁴, which also express Nap1l2.

We cloned the coding sequence of Nap1l2 into the pEGFP-Cl vector (Clontech) allowing expression of GFP-fusion proteins under the control of the CMV promoter. Upon transfection of subconfluent P19 cell cultures, NAP1L2 localizes either to the cytoplasm or to both the nucleus and the cytoplasm. Cell cycle arrest experiments showed that the protein is cytoplasmic in the G1 phase (FIG. 11a), whereas localization in the nucleus occurs in cells that enter S-phase (FIG. 11b). DAPI staining indicated co-localisation of NAP1L2 in regions of the nucleus with high chromatin density. Interestingly, when cells expressing NAP1L2 were kept subconfluent and growing, almost all of the NAP1L2 positive cells became apoptotic and died within 24 hours. In the apoptotic cells, the NAP1L2 protein remained associated with the fragmented nuclei (FIG. 11c). NAP1L2 expressing cells arrested in G1 phase in contrast survived in culture and few of these cells became apoptotic. Similar results were observed on overexpression of the unfused NAP1L2 protein, but not on overexpression of GFP alone (data not shown). When placed under the control of the Nap1l2 promoter, GFP-NAP1L2 similarly localized to the cytoplasm during G1 phase. Cells expressing NAP1L2 in the nucleus after replication showed condensation of the replicated chromatin, often associated with apoptosis (FIGS. 11d, e, f, g).

Our data show the importance of Nap1l2 in the process of neurulation. Deletion of Nap1L2 leads to embryonic lethality and the neural tube closure defects found from mid-gestation onwards closely resemble spina bifida and/or anencephaly in humans¹⁵. The severity of the mutant phenotype correlates with the extent of chimerism as demonstrated by lacZ expression. Differentiation studies in vitro confirmed that overproliferation of neuronal precursor cells is associated with the mutant phenotype and suggests that NAP1L2 may be implicated in the control of cell division.

The following Methods were employed in carrying out the Examples.

In situ hybridization. The experiments were performed using the method described in Young et al.³² modified according to Le Novere et al.³³. The sequence of the 45mer oligonucleotide used, 5′-

| | |
|---|---|
| 5′-TTATCACAGTCACATACAATCAGAAGCCTTGCACTAGCTGTTATC-3′ | (SEQ ID NO:5), |

was chosen from the 3′ untranslated region of Nap1l2. The temperature of stringent rinse step was 45° C. The specificity of the labeling was verified in control experiments where the labeled oligonucleotide was displaced by an excess of unlabeled probe.

Construction of the knockout ES cells. Nap1l2 cDNA was used to screen a 129/Sv genomic phage library in Lambda Dash I I (Stratagene). Six phages were identified and analyzed by single and double restriction digests with eight different enzymes. The restriction pattern of the phage DNAs was compared to that of genomic DNA. The insert of one of the phages was subcloned into pBluescript SK(+) (Stratagene) using the restriction enzymes Notl and XhoI. A deletion in the Nap1l2 gene was generated using the enzyme HincII followed by intramolecular relegation. This eliminated 890 bp coding sequence. Relegation created a unique SalI site, which was then used to insert in phase a β-galactosidase reporter gene and a neomycin resistance gene. The cloning of these cassettes disrupted the reading frame of the remaining Nap1l2 sequence. Finally, a HSV-tk cassette was inserted into the polylinker of the vector (FIG. 2). The knockout construct was transfected into the embryonic stem cell line CK35 (a male 129/Sv cell line obtained from Dr. Babinet, Institut Pasteur), and transfected clones were selected with geneticin (G418) and ganciclovir. Southern blot analysis for screening of putative recombinants was optimized using the E18 single copy probe flanking the construct, which was isolated from overlapping lambda clones³⁴. Two clones showed the expected restriction pattern for correct integration of the βGalNeo-cassette into the Nap1l2 gene. Both clones were used for microinjection into C57BL/6 blastocysts³⁵ and morula aggregation experiments using either C57BL/6 or CD-1 morulas.

Morula aggregation. Experiments were performed according to the protocols of Wood et al.³⁶ and Nagy et al.³⁷. Embryos were obtained from three to five week old females injected IP with PMSG (5 IU) then 46 hours later with hCG(5 IU), and immediately mated to stud males. Females were checked for plugs the following morning, noon of this day being considered as E0.5. Positive females were sacrificed E2.5 by cervical dislocation and the female reproductive track isolated into PBS. Morulas were isolated by flushing oviducts using a blunt 30 gauge needle and M2 media. Zona pelucidas were removed by brief incubation in acid Tyrodes. Compacted and non-compacted morulas were then transferred singly or in pairs into wells of tissue culture dishes containing M16 media under mineral oil. Feeder-free, ES cell clumps (5-8 ES cells) from partially trypsinized ES cell colonies were added to wells containing morulas and incubated for 4 hours in a 37° C., 5% CO₂ humidified incubator. ES cell/morula aggregates were then transferred to fresh tissue culture dishes containing M16 media under mineral oil and incubated overnight. Expanded blastocysts were implanted into the uterine horns of anaesthetized E2.5 foster mothers, which had been mated to vasectomized males.

Whole mount X-Gal staining of embryos. The X-Gal staining of embryos was essentially performed as described in Papenbrock et al.³⁸. This procedure is ideally suited for day E10.5 embryos, for older embryos incubation times were lengthened accordingly. Embryos were dissected into PBS. The PBS was then replaced by a solution of 0.5% gluteraldehyde in PBS and the embryos shaken for 30 minutes at RT. After three successive rinses with PBS for a total of 30 minutes, the PBS was replaced by the staining solution (PBS containing 10 mM potassium ferrocyanide, 10 mM potassium ferricyanide, I mM spermidine, 2 mM MgCl₂, 0.02% NP-40, 0.01% sodium deoxycholate, and 0.05% X-Gal). Overnight incubation was at 30° C., followed by rinsing in PBS and fixation in 3.7% formaldehyde in PBS for several hours.

Histological survey. The embryos were either frozen in dry-ice powder, sectioned on cryostat (14 μm thick sections), mounted on Superfrost+ slides and stored at −80° C., or dehydrated immediately through an alcohol gradient, immersed in xylene, xylene/paraffin (1:1), and embedded in paraffin before microtome sectioning. X-Gal staining of frozen sections was performed following the protocol in Hogan et al.³⁹. All sections were counterstained with either hematoxylin or toluidine blue. After dehydration through ethanol series, the slides were immersed in Histoclear (Prolabo) and mounted Eukitt (Prolabo).

Differentiation of ES cells. Differentiation in vitro was carried out following the protocol described in Fraichard et al.¹⁸.

Immunofluorescence. Fixation was done for 20 minutes in 2% paraformaldehyde, followed after a rinse in PBS, by permeabilization with 0.02% Triton X-100 for two minutes. After three rinses of five minutes in PBS, the slides were incubated for one hour at 37° C. with the diluted antisera. Dilutions were in PBS: anti-β-tubulin III (1/400) Sigma T-8660), anti-GFAP (1/200) (DAKO Z03345), anti-NF200 (1/200) (Sigma N-4142), and anti-nestin antibody (Rat-401 from the Developmental Studies Hybridoma Bank, University of Iowa) (1/200 dilution). Secondary FITC-labeled antibodies were from Sigma and used in 1/300 dilutions After rinsing in PBS as before, slides were mounted in 2% n-propylgallate in glycerol. LacZ staining was performed after fixation and 3 rinses in PBS as described above.

Our invention thus includes the following findings:

A) Analysis of the Human NAP1L2 Gene in NTD Patients

We analysed X-linked familial and spontaneous cases of NTD for sequenced alterations in the human NAP1L2 gene. No differences were found in the familial cases available for study. On the other hand, a number of single nucleotide polymorphisms (SNPs) were identified within the 5′ region of NAP1L2 in both cases of spontaneous NTD and in normal controls. Most of these SNPs lead to the replacement of guanidines or cytosines within a CpG island that is conserved between the human and the mouse promoter regions. Demethylation with 5 azacytidine in vitro activates NAP1L2 transcriptional activity suggesting the importance of CpG methylation/demethylation in regulating the activity of the Nap1L2/NAP1L2 genes in neuronal cells and the potential importance of the polymorphisms in modifying the transcriptional activity Nap1L2/Nap1l2 expression may depend on epigenetic-environmental factors such as folic acid levels that can influence DNA methylation and that is frequently associated with neural tube closure defects.

B) Interactions of the Nap1l2 Protein

Our work has revealed that the Nap1l2 protein interacts with histones that are integrated in the chromatin during DNA replication in the S-phase of the cell cycle. In differentiating neurons, Nap1l2 remains associated with these histones and the chromatin suggesting a role for Nap1l2 in chromatin remodeling and cell type specific gene regulation. We suggest that Nap1l2 is likely to be required for establishing or maintenance of the postmitotic stage of neurons. Our results clearly suggest a putative role for Nap1l2 in neuronal degeneration and/or regeneration processes.

C) NAP1L2 in Tumours

Our recent results have shown that NAP1L2 is expressed in human neuroblastoma tumour cells.

As used herein, the term “heterologous amino acid sequence” means a sequence which does not immediately follow NAP1L2 or Nap1L2 promoter in the nature.

As used herein, the term “heterologous polypeptide” means a polypeptide which is encoded by a sequence which does not immediately follow NAP1L2 or Nap1L2 promoter in the nature and which expression is not under NAP1L2 or Nap1L2 promoter control in the nature NAP1L2 or Nap1L2 promoter.

### REFERENCES

The following publications have been cited herein. The entire disclosure of each publication is relied upon and incorporated by reference herein.

1. Kerszberg., M. & Changeux, J.-P. A simple molecular model of neurulation. BioEssays 20, 758-770 (1998).

2. Wilson, P. A. & Hemmati-Brivanlou, A. Vertebrate neural induction: Inducers, inhibitors, and a new synthesis. Neuron 18, 699-710 (1997).

3. Emery, A. E. H. in Methodology in Medical genetics (ed 2) 58 (Churchill Livingstone, Edinburgh 1986).

4. Echelard, Y. et al. Sonic hedgehog, a member of a family of putative signaling molecules, is implicated in the regulation of CNS polarity. Cell 75, 1417-30 (1993).

5. Strachan, T. & Read, A. P. PAX genes. Curr. Opin. Genet. Dev. 4, 427-38 (1994).

6. Edelman, G. M., Gallin, W. J., Delovee, & Cunningham, B. A. Early epochal maps of two different cell adhesion molecules. Proc. Natl. Acad. Sci. USA 80, 4384-4388 (1983).

7. Vogel, K. S. & Parada, L. F. Sympathetic neuron survival and proliferation are prolonged by loss of p53 and neurofibromin. Mole Cell Neurosci. 11, 19-28 (1998).

8. Altman, R. & Kellog, D. Control of mitotic events by Napl and Gin4 kinase. J. Cell Biology 138, 119-130 (1997).

9. Simon, H. U. et al. Molecular characterization of hNRP, a cDNA encoding a human nucleosome-assembly-protein-I-related gene product involved in the induction of cell proliferation. Biochem. J. 297, 389-97 (1994).

10. Laskey, R. A., Honda, B. M., Mills, A. D. & Finch, J. T. Nucleosomes are assembled by an acidic protein which binds histones and transfers them to DNA. Nature 275, 416-420 (1978).

11. Ito, T., Bulger, M., Kobayashi, R. & Kadonaga, J. T. Drosophila NAP-1 is a core histone chaperone that functions in ATP-facilitated assembly of regularly spaced nucleosomal arrays. Mol. Cell Biol. 16, 3112-24 (1996).

12. McQuibban, G. A., Commisso-Cappelli, C. N. & Lewis, P. N. Assembly, remodeling, and histone binding capabilities of yeast nucleosome assembly protein 1. J. Biol. Chem. 273,6582-90 (1998).

13. Walter, P. P., Owen-Hughes, T. A., Cote, J. & Workman, J. L. Stimulation of transcription factor binding and histone displacement by nucleosome assembly protein 1 and nucleoplasmin requires disruption of the histone octamer. Mol. Cell Biol. 15, 6178-87 (1995).

14. Rodriguez, P. et al. Functional characterization of human nucleosome assembly protein-2 (NAP1L4) suggests a role as a histone chaperone. Genomics 44, 253-65 (1997).

15. Kellogg, D. R., Kikuchi, A., Fujii , Nakata, T., Turck, C. W. & Murray, A. W. Members of the NAP/SET family of proteins interact specifically with B-type cyclins. J. Cell Biol. 130, 661-73 (1995).

16. Rougeulle, C. & Avner, P. Cloning and characterization of a murine brain specific gene Bpx and its human homologue lying within the Xic candidate region. Hum. Mol. Genet. 5, 41-49 (1996).

17. Watanabe, T. K. et al. Cloning, expression pattern and mapping to Xq of NAP1L3, a gene encoding a peptide homologous to human and yeast nucleosome assembly proteins. Cytogenet. Cell Genet. 74, 281-5 (1996).

18. Fraichard, A. et al. In vitro differentiation of embryonic stem cells into glial cells and functional neurons. J. Cell Sci. 108, 3181-3188 (1995).

19. Struebing, C. et al. Differentiation of pluripotent embryonic stem cells into the neuronal lineage in vitro gives rise to mature inhibitory and excitatory neurons. Mech. Dev, 53,275-287 (1995).

20. Kalcheim, C., Carmeli, C. & Rosenthal, A. Neurotrophin 3 is a mitogen for cultured neural crest cells. Proc. Natl. Acad Sci. USA 89, 1661-5 (1992).

21. Rhinn, M., Dierich, A., Le Meur. M. & Ang, S.-L. Cell autonomous and non-cell autonomous functions of Otx2 in patterning the rostral brain. Development 126,4295-4304 (1999).

22. Ishibashi, M. et al. Targeted disruption of mammalian hairy and Enhancer of split homolog-1 (HES-1) leads to up-regulation of neural helix-loop-helix factors, premature neurgenesis, and severe neural tube defects. Genes & Dev. 9, 3136-3148 (1995).

23. Wilson, D. B. Proliferation in the neural tube of the Splotch (Sp) mutant mouse. J. Comp. Neurol. 154, 249-256 (1974).

24. Bennett, G. D. et al. Neurulation abnormalities secondary to altered gene expression in neural tube defect susceptible Splotch embryos. Teratology 57, 17-29 (1998).

25. Keller-Peck, C. R. & Mullen, R. J. Altered cell proliferation in the spinal cord of mouse neural tube mutants curly tail and Pax3 splotch-delayed. Brain. Res. Dev. Brain Res. 102, 177-88 (1997).

26. Hol, F. A. et al. PAX genes and human neural tube defects: an amino acid substitution in PAX1 in a patient with spina bifida. J. Med. Genet. 33, 655-660 (1996).

27. Hol, F. A. et al. Molecular genetic analysis of the gene encoding the trifunctional enzyme MTHFD (methylenetetrahydrofolate-dehydrogenase, methylenetetrahydrofolate-cyclohydrolase, formyltetrahydrofolate synthetase) in patients with neural tube defects. Clin. Genet. 53, 119-125 (1998).

28. Vargas, F. R. et al. Analysis of the human Sonic Hedgehog coding and promoter regions in sacral agenesis, triphalangeal thumb, and mirror polydactyly. Hum. Genet. 102, 387-92 (1998).

29. Jensson, O. et al. A family showing apparent X linked inheritance of both anencephaly and spina bifida. J. Med. Genet. 25, 227-229 (1988).

30. Moore, G. E. et al. X chromosome genes involved in the regulation of facial clefting and spina bifida. Cloft Palate J. 27, 131-135 (1990).

31. Neumann, P. E. et al. Multifactorial inheritance of neural tube defects: localization of the major gene and recognition of modifiers in ct mutant mice. Nat. Genet. 6, 357-62 (1994).

32. Copp, A J., Checiu, I. & Henson J. N. Developmental basis of severe neural tube defects in the loop-tail (Lp) mutant mouse: Use of microsatellite DNA markers to identify embryonic genotype. Dev. Biol. 165, 20-29 (1994).

33. Young W. S. 3d , Bonner T. I. & Brann, M. R Mesencephalic dopamine neurons regulate the expression of neuropeptide mRNAs in the rat forebrain. Proc. Natl. Sci. USA 83, 9827-31 (1986).

34. Le Novere, N., Zoli, M. & Changeux, J. P. Neuronal nicotinic receptor alpha 6 subunit MRNA is selectively concentrated in catecholaminergic nuclei of the rat brain. Eur. J. Neurosci. 8, 2428-39 (1996).

35. Rougeulle, C., Colleaux, L., Dujon, B. & Avner, P. Generation and characterization of an ordered lambda clone array for the 460-kb region surrounding the murine Xist sequence. Mamm. Genome 5, 416-23 (1994).

36. Gardner, R. L. Mouse chimeras obtained by the injection of cells into the blastocyst. Nature 220, 596-7 (1968).

37. Wood, S. A., Pascoe, W. S., Schmidt, C., Kemler, R., Evans, M. J. & Allen, N. D. Simple and efficient production of embryonic stem cell-embryo chimeras by coculture. Proc. Natl. Acad. Sci. USA 90, 4582-5 (1993).

38. Nagy, A., Rossant, J., Nagy, R., Abramow-Newerly, W. & Roder, J. C. Derivation of completely cell culture-derived mice from early-passage embryonic stem cells. Proc. Natl. Acad. Sci. USA 90, 8424-8 (1993).

39. Papenbrock, T., Peterson, R. L., Lee, R. S., Hsu, T., Kuroiwa, A. & Awgulewitsch, A. Murine Hoxc-9 gene contains a structurally and functionally conserved enhancer. Dev. Dyn. 12, 540-7 (1998).

40. Hogan, B., Beddington, R., Costantini, F. & Lacy, E. in Manipulation the mouse embryo 373-375 (ColdSpring Harbor Laboratory Press New York 1994).

What is claimed is:

1. A method for screening neural system defects in a mammal, said method comprising:
(A) providing chromosomal material from said human;
(B) detecting a modification of the NAP1L2 gene in the chromosomal material, wherein said modification is selected from a) substitution, b) deletion, c) frame-shift, d) insertion aberent or e) altered epigenetic control; that causes a loss of biological function in the NAP1L2 gene; and
(C) correlating the modification of said gene with a potential for a neural system defect.

2. A method according to claim 1 where the said screening of neural system defects concerns a human being.

3. The method of claim 1, wherein said modification in the NAP1L2 gene is detected by hybridization with a labeled probe.

4. The method of claim 3, wherein said probe is a oligonucleotide probe of SEQ ID NO:3.

5. A method of claim 1, wherein said modification is detected by
(A) amplification of the chromosomal material using PCR;
(B) sequencing said material to detect the modification of the nucleotide sequence; and
(C) correlating the modification of said gene with a potential for neural system defects.

6. A method for screening neural system defects in a human, said method comprising:
(A) providing biological material from said human;
(B) detecting the absence, inappropriate, or modified expression of NAP1L2 gene product using labeled antibodies to said gene product; and
(C) correlating said absence, inappropriate, or modified expression with a potential for neural system defects.

7. The method of claim 5, wherein the said antibodies are polyclonal.

8. The method of claim 5, wherein the said antibodies are monoclonal.

9. A method of any one of claims 1 to 8, wherein the neural system defect results from a failure of or incomplete neural tube closure.

10. A method of claim 9, wherein said incomplete neural tube closure results in spina bifida.

11. The method of any one of claims 1 to 8, wherein the neural system defect results from inappropriate control of nucleosome activity in neurons.

12. The method of any one of claims 1 to 8, wherein the neural system defect results from inappropriate control of the cell cycle in neurons.

13. The method of any one of claims 1 to 7, wherein the neural system defect results from inappropriate differentiation of neurons.

14. The method of any one of claims 1 to 7, wherein the neural system defect results from inappropriate maintenance of neurons.

15. A recombinant polynucleotide comprising a nucleotide sequence, wherein said sequence includes at least one modification of the NAP1L2 gene, wherein said modification is selected from a) substitution, b) deletion, c) frame-shift, d) insertion, or e) site-directed mutagensis that causes a loss of biological function in the NAP1L2 gene.

16. A recombinant polynucleotide comprising a nucleotide sequence including at least the NAP1L2 gene and its promoter included in SEQ ID NO:4.

17. The polynucleotide of claim 15 or 16, wherein said polynucleotide is a chromosome or a part of it of a neural cell.

18. A neural cell containing the polynucleotide of claim 17.

19. The neural cell of claim 18, wherein the cell is derived from an immortal cell line, such as embryonic stem cells, neuronal cell line, or tumor derived cell line.

20. The neural cell of claim 18, wherein the NAP1L2 gene is under control of a neural-specific promoter, such as nestin, other neuronal members, and inducible promoters.

21. The neural cell of claim 17 or 19, wherein the neural cell is from a wild-type animal.

22. The neural cell of claim 18, 20, or 21, wherein the NAP1L2 gene is modified, wherein said modification is selected from a) substitution, b) deletion, c) frame-shift, d) insertion, or e) site-directed mutagenesis that causes a loss of biological function in the NAP1L2 gene.

23. The neural cell of claim 18, 20, or 21, wherein the NAP1L2 gene is altered through a naturally occurring mutation.

24. A polynucleotide comprising the promoter of the NAP1L2 gene in SEQ ID NO:4, or a polynucleotide hybridizing under stringent conditions with SEQ ID NO:4, or at least 20 nucleotides of said SEQ ID NO: 4.

25. A vector containing the polynucleotide of claim 24.

26. A method of making a recombinant neural cell comprising:
(A) providing a neural cell;
(B) modifying a NAP1L2 gene or the promoter of the NAP1L2 gene in the neural cell, wherein said modification is selected from a) substitution, b) deletion, c) frame-shift, and d) insertion that causes a loss of biological function in the gene; and
(C) selecting modified cells.

27. A method of screening for therapeutic compounds comprising:
(A) providing a cell of any one of claims 18 to 23;
(B) introducing to the cell a compound to be screened; and
(C) correlating change in the proliferation of cells with the activity of the compound.

28. A method of screening for therapeutic compounds comprising:
(A) providing a transgenic knockout animal containing the human NAP1L2 gene in its chromosomes.
(B) introducing to the animal a compound to be screened; and
(C) correlating a change in the development and maturation of the nervous system with the activity of the compound.

29. A method for screening neural system defects in a mouse, said method comprising:
(A) providing chromosomal material from said mouse;
(B) detecting a modification of the Nap1l2 gene in the chromosomal material, wherein said modification is selected from a) substitution, b) deletion, c) frame-shift, or d) insertion that causes a loss of biological function in the Nap1l2 gene;
(C) correlating the modification of said gene with a potential for a neural system defect.

30. A method for screening neural system defects in a mouse, said method comprising:
(A) providing biological material from said mouse;
(B) detecting the absence, inappropriate, or modified expression of Nap1l2 gene product using labeled antibodies to said gene product; and
(C) correlating said absence or inappropriate expression with a potential for neural system defects.

31. A recombinant polynucleotide comprising a nucleotide sequence, wherein said sequence includes at least one modification of the Nap1l2 gene, wherein said modification is selected from a) substitution, b) deletion, c) frame-shift, d) insertion, or e) site-directed mutagenesis that causes a loss of biological function in the Nap1l2 gene.

32. A polynucleotide comprising the promoter of the Nap1l2 gene in SEQ ID NO:1, or a polynucleotide hybridizing under stringent conditions with SEQ ID NO: 1, or at least 20 nucleotides of said SEQ ID NO: 1.

33. A vector containing the nucleic acid molecule of claim 32.

34. A recombinant neural cell comprising a vector comprising the Nap1l2 gene.

35. The neural cell of claim 34, wherein the Nap1l2 gene is under control of a neural-specific promoter, such as nestin or other neuronal genes or inducible promoters.

36. A recombinant neural cell of claim 35, wherein the Nap1l2 gene of the native cell is modified, wherein said modification is selected from a) substitution, b) deletion, c) frame-shift, d) insertion, or e) site-directed mutagenesis that causes a loss of biological function in the Nap1l2 gene.

37. A method of screening for therapeutic compounds comprising:
(A) providing a cell containing a polynucleotide according to claim 32;
(B) introducing to the cell a compound to be screened; and
(C) correlating change in the proliferation of the cell with the activity of the compound.

38. The method of claim 37, wherein said change in proliferating cells is a control of cancer of neural cells.

39. Use of therapeutic compounds obtained by the method according to claim 27 for increasing the expression of NAP1L2 gene in tumoral human neural cells or for decreasing the expression of NAP1L2 gene in human neural cells afflicted by a degenerating disease.

40. An eukaryotic cell containing the insert comprised in the plasmid pBPX1 or pBPX2 or pBPX3 or polynucleotide hybridizing under stringent conditions with the said insert.

41. A plasmid consisting in the deposit made at C.N.C.M. under the Accession Number I-2463.

42. A plasmid consisting in the deposit made at C.N.C.M. under the Accession Number I-2464.

43. A plasmid consisting in the deposit made at C.N.C.M. under the Accession Number I-2465.

44. A plasmid consisting in the deposit made at C.N.C.M. under the Accession Number I-2466.

45. A polynucleotide containing the sequence SEQ ID NO:6
(SEQ ID NO. 6)
Genomic sequence BPX human

| | |
|---|---|
| 1. | acttaaaggaaaaatttatctataaactgacagaatttagaaataaatacaacaatatgtaaacagttttaatatctgtg |
| 2. | atagtaacaaattctttaaatctggaaaataatagtcacttaaaattttaaaaaattgttcaattaataaatgatccaag |
| 3. | ttagaaatatgaacaaaataaacctcaccaataattactatagagaggaaattttaattactgcaaagctttccatccta |
| 4. | taaatacattatcaaatagtttaaccatttctttaatgctgagatttagattatttccaattaactcaaaagcatcaagc |
| 5. | aaatgttatgatttctaagaataaacataactttccattttggcttttgtatatatgtatatttctaacggctgttaaag |
| 6. | ccagcattaagaaggagaagcagaaagtcagtattgggactggggttatttataagccaggcaactggttaattgtggtt |
| 7. | aattgtctggtatgtttactagtcacgtagttgtatacaccatactagtttttcatcacaggccctcattcgcccccact |
| 8. | gccatcggacttcctcctcctcccctcacaggaaatgtttcgagaatttttcaacctaaaatcatatagcttgtgaaaaa |
| 9. | taccgacaaacataatatagaatatttaaataactgacacgccacctaaagaccatcagtgctaattcctggtgttttta |
| 10. | atctttgaagcgtttgtttatcagctcttccaccatccacctgtcccctccccaggtccccgatctaaaatcaaagagat |
| 11. | tgatttaggatgggtgggtgccttgtcttctctcattgttcgacattttagttacgttttctctgagctctctggaaagc |
| 12. | ataaaagtataatatctgttaaaagttggatgaatgaactaatgaacgcaatgggattccagaaaactctgcgggagatg |
| 13. | ggctagaggacgaggaggaggtggatgaatcagccatgttagagagcctgggaaggtgagcagagttgaaaacttgatag |
| 14. | atctaataatttactggctctgggtttgtcagtcactacattgcagcaaatgagattagagcatagttgtgggagggaag |
| 15. | gaggtgacgcagcaatctatttgcacctagaaattttaggcaagtgatagctgcgtaatcatactgcggcaccgtttttt |
| 16. | tcttgcagcagtagctgcttgcggaggaggtctgcccactgcagctctctgcagtctccggctctctcctgcaggatcgg |
| 17. | tcaacgcagccgtcgccgccctctgcacccagcccaggtcgccactgcttcagtccggttctcaaagcctcagcaccatc |
| 18. | ttttatccccgagcagcctggatcgtcgttccctcagtccggacgccactgctaggtccgaccaccgccacttctgatat |
| 19. | ttcggtgagtcttttcctgtggaggtttggtctcccgatctctgtggtagccaccttaggcgtgtacggtcctttgaaaa |
| 20. | ATGGCCGAGTCAGAGAACCGCAAGGAGCTGTCAGAATCCAGTCAAGAAGAGGCTGGTAATCAGATAATGGTGGAAGGGCT |
| 21. | CGGGGAACATCTGGAGCGCGGTGAAGATGCCGCTGCTGGGCTTGGAGACGATGGGAAGTGCGGTGAAGAAGCTGCCGCTG |
| 22. | GGCTTGGGGAAGAAGGGGAAAACGGTGAAGATACTGCTGCTGGGTCCGGGGAAGATGGGAAAAAAGGTGGCGATACTGAT |
| 23. | GAGGACTCAGAGGCAGACCGTCCAAAAGGACTTATCGGTTATGTTTTAGATACAGACTTTGTTGAAAGTCTACCTGTGAA |
| 24. | AGTTAAGTACCGTGTGTTAGCCCTTAAAAAGCTTCAAACTAGAGCGGCCAATTTAGAATCCAAATTCCTGAGGGAATTTC |
| 25. | ATGACATTGAAAGAAACTTTGCTGAAATGTACCAACCCTTACTGGAAAAAAGACGTCAGATCATCAATGCAATCTATGAA |
| 26. | CCTACAGAAGAGGAATGTGAATATAAATCAGACTCTGAGGACTCTGATGATGAGGAAATGTGTCATGAAGAGATGTATGG |
| 27. | TAATGAGGAGGGTATGGTACATGAATATGTGGATGAGGACGATGGTTATGAGGACTATTATTATGATTATGCTGTGGAAG |
| 28. | AGGAGGAGGAGGAGGAGGAGGAGGACGACATTGAGGCTACTGGAGAAGAGAATAAAGAAGAGGAGGATCCTAAGGGAATT |
| 29. | CCTGATTTTTGGCTAACTGTTTTAAAAAACGTTGATACACTCACTCCTTTGATTAAGAAATATGATGAGCCTATTCTGAA |
| 30. | GCTCCTGACAGATATTAAAGTTAAGCTTTCAGATCCTGGCGAGCCCCTCAGTTTCACACTAGAATTTCACTTCAAACCCA |
| 31. | ATGAATATTTCAAAAATGaGTTGTTGACAAAGACCTATGTGCTGAAGTCAAAGCTAGCATATTATGATCCCCATCCCTAT |
| 32. | AGGGGAACTGCGATTGAGTATTCCACAGGCTGTGAGATAGATTGGAATGAAGGAAAGAATGTCACTTTGAAAACCATCAA |
| 33. | GAAGAAACAGAAACATCGGATCTGGGGAACAATCCGAACTGTAACTGAAGATTTTCCCAAGGATTCATTTTTCAATTTTT |
| 34. | TCTCTCCTCATGGAATCACCTCAAATGGAAGGGATGGAAATGATGATTTTTTACTTGGTCACAATTTACGTACTTACATA |
| 35. | ATTCCAAGATCAGTATTATTTTTCTCAGGTGATGCACTGGAATCTCAGCAGGAGGGGGTAGTTAGAGAAGTTAATGATGC |
| 36. | AATTTATGACAAAATTATTTATGATAATTGGATGGCTGCAATTGAGGAACTTAAAGCTTGTTGCAAAAACCTTGAGGCAT |
| 37. | TAGTAGAAGACATTGATCGTTAGAGCagagtatacatggccctgaaattaactgccctagatatagttactcaagctata |
| 38. | agaagccttgtgttctgtattttgctttgtagtgttagttaaaacatatgtttcaaaaatataagaaaagttcaaaaact |
| 39. | aattaatttgaccttgagttttagtagtagaatgttttcaagaaatgtacactgtggtaaatgatttaaaacactagtat |
| 40. | agtgttgtgtagcttaatccttctgaagtctttttgtcatgtagctattaatctgtggctatgaaatgatcagaaatgct |
| 41. | aagtgagatcaatatttgtttggaaaaaaaatcttgggaaacaacccaagggttttcgctgttgttgtttttctttttct |
| 42. | atttttgtttacttagtcctttagctagtggatttaattttgttgtgcctgcttcattttgcaataacaatgcagtagaa |
| 43. | tttaaaacttggatgcttaagaggcctgcatatagataagaatttcaggcaaaactacatttattgttaataacagcttg |
| 44. | ttcataggctcttgtattttatgtaactgtgataaataatgaaaacttagttatattgaggttattgtttgtcggtgaag |
| 45. | tgttagtcacagtattttcaaaagtttgcacatattgttctgtgtaattgtgtaagccataattacagtgtttaattctc |
| 46. | ttttcctattacatcattcattgaaagtgatcactttaccattttgaaaagatatttcgtgttctttcactgcaaaataa |
| 47. | aaagaataaaaatttcaga |

46. The polynucleotide of claim 24, wherein said polynucleotide further comprises an heterologous amino acid sequence coding for an heterologous polypeptide under the control of NAP1L2 promoter.

47. A vector containing the polynucleotide of claim 46.

48. A neural cell containing the polynucleotide of claim 46.

49. A process for targeted expression of a polypeptide in a neural cell wherein said neural cell is a cell according to claim 48.

50. The polynucleotide of claim 32, wherein said polynucleotide further comprises an heterologous amino acid sequence coding for an heterologous polypeptide under the control of Nap1l2 promoter

51. A vector containing the polynucleotide of claim 50.

52. A neural cell containing the polynucleotide of claim 50.

53. A process for the targeted expression of a polypeptide in a neural cell wherein said neural cell is a cell according to claim 52.
